(19) United States
(12) Patent Application Publication
(10) Pub. No.: US 2016/0015789 A1
(43) Pub. Date: Jan. 21, 2016

(54) FORMULATIONS OF AN ALBUMIN hGH FUSION PROTEIN

(71) Applicants: Jason BOCK, North Potomac, MD (US); Yen-Huei LIN, Rockville, MD (US); Ping FENG, Germantown, MD (US)

(72) Inventors: Jason BOCK, North Potomac, MD (US); Yen-Huei LIN, Rockville, MD (US); Ping FENG, Germantown, MD (US)

(73) Assignee: TEVA PHARMACEUTICAL INDUSTRIES, LTD., Petach-Tikva (IL)

(21) Appl. No.: 14/801,639

(22) Filed: Jul. 16, 2015

Related U.S. Application Data
(60) Provisional application No. 62/025,957, filed on Jul. 17, 2014.

(51) Int. Cl. A61K 38/27; A61K 47/26; C07K 14/61; A61K 47/34; A61K 38/38; C07K 14/765; A61K 47/22; A61K 47/02

(52) U.S. Cl. CPC A61K 38/27; A61K 47/22; A61K 47/26; A61K 47/02; A61K 47/34; A61K 38/385; C07K 14/765; C07K 14/61; C07K 2319/31

(57) ABSTRACT

A stable liquid pharmaceutical composition comprising an albumin-human growth hormone (hGH) fusion protein whose amino acid sequence is set forth as SEQ ID NO: 1 and a buffer, wherein the stable liquid pharmaceutical composition has a pH range of 5.5-6.5.

This application claims the priority of U.S. Provisional Application No. 62/025,957, filed Jul. 17, 2014, the contents of which are hereby incorporated by reference.

Throughout this application, various publications are referenced, including referenced in parenthesis. Full citations for publications referenced in parenthesis may be found listed at the end of the specification immediately preceding the claims. The disclosures of all referenced publications in their entireties are hereby incorporated by reference into this application in order to more fully describe the state of the art to which this invention pertains.

### BACKGROUND OF THE INVENTION

Growth hormone (GH) is a pituitary hormone that has the primary function of growth promotion. In children, GH promotes linear growth by regulating the endocrine and paracrine production of insulin-like growth factor I (IGF-I), which is produced by the liver and other target tissues, including the epiphyseal growth plate (Emedicine 2012). The non-growth promoting effects of GH relate to its role in metabolism. Recombinant protein drugs, such as recombinant human GH (rhGH) used for replacement therapy for GH deficient children and adults, have become a cornerstone of medical and especially endocrine practice (Saenger 2009).

However, pharmaceutical formulations of growth hormone have a tendency to be unstable (Johnson 1989). Solutions of growth hormone are prone to generate degradation products such as deamidated or sulfoxylated products and dimer or polymer forms, especially in solution. hGH is known to become deamidated upon in vitro aging at weakly alkaline pH and deamidation of asparagine side chains at specific sites is a major contributor to degradation of hGH, particularly when in solution (Johnson 1989, Ablinger 2010, Robinson 2001). Studies have shown that the primary site of deamidation of rhGH occurs at the asparagine in position 149 and is coupled with the isomerization of the aspartate in position 130, forming isoaspartate (Johnson 1989, Teshima 1991).

ALBUTROPIN™ (also referred to as TV-1106) is an albumin-human growth hormone (hGH) fusion protein comprising a single polypeptide composed of the mature form of human serum albumin (HSA) (residues 1-585) fused at its C-terminus to the N-terminus of the mature form of hGH (residues 586-776). ALBUTROPIN™ retains pharmacologic activity of GH in vivo while offering a substantially longer duration of action than recombinant GH alone (US 2014-0162954 A1, Osborn 2002, Chou 2005). U.S. Patent Application Publication No. US 2014-0162954 A1 describes a lyophilized formulation of ALBUTROPIN™ which is reconstituted with buffer before administration. Reconstitution presents complications and risks such as contamination and incorrect dosing. Moreover, the requirement for reconstitution can reduce patient compliance. Thus, storage stable liquid formulations of ALBUTROPIN™ which do not require reconstitution by the patient are needed.

### SUMMARY OF THE INVENTION

The invention provides a stable liquid pharmaceutical composition comprising: an albumin-human growth hormone (hGH) fusion protein whose amino acid sequence is set forth as SEQ ID NO: 1 a buffer, wherein the stable liquid pharmaceutical composition has a pH of 5.5-6.5.

The invention also provides a stable liquid pharmaceutical composition comprising (i) an albumin-human growth hormone (hGH) fusion protein whose amino acid sequence is set forth as SEQ ID NO: 1, and (ii) a pharmaceutically acceptable excipient, in which 0.01-0.15 moles of isoaspartate residues are present per mole of fusion protein after storage for 12 months at 2-8° C.

The invention also provides a stable liquid pharmaceutical composition comprising (i) an albumin-human growth hormone (hGH) fusion protein whose amino acid sequence is set forth as SEQ ID NO: 1, and (ii) a pharmaceutically acceptable excipient, in which 1-5% of the fusion protein has an isoaspartate residue at amino acid position D715 after storage for 6 months at 2-8° C.

The invention provides a package comprising any of the stable liquid pharmaceutical compositions of the invention and a container.

The invention also provides a process for preparing a stable liquid pharmaceutical composition comprising an albumin-human growth hormone (hGH) fusion protein whose amino acid sequence is set forth as SEQ ID NO: 1, the process comprising (a) determining the number of moles of isoaspartate residues present per mole of fusion protein in a batch of the fusion protein; and (b) preparing the stable liquid pharmaceutical composition from the batch only if less than 0.05, less than 0.04, less than 0.03, or less than 0.02 moles of isoaspartate residues are present per mole of fusion protein in the batch.

The invention also provides a process for preparing a stable liquid pharmaceutical composition comprising an albumin-human growth hormone (hGH) fusion protein whose amino acid sequence is set forth as SEQ ID NO: 1, the process comprising (a) determining the percentage of fusion protein having an isoaspartate residue at least one of amino acid positions D715, N734, N684, D692, or D697 in a batch of fusion protein; and (b) preparing the pharmaceutical composition from the batch only if the percentage of fusion protein having an isoaspartate residue at the one or more amino acid positions determined in step (a) is below a threshold percentage predetermined for the at least one of amino acid positions D715, N734, N684, D692, or D697.

The invention also provides a process for validating a batch of a stable liquid pharmaceutical composition comprising an albumin-human growth hormone (hGH) fusion protein whose amino acid sequence is set forth as SEQ ID NO: 1 for distribution, the process comprising (a) determining the number of moles of isoaspartate residues present per mole of fusion protein in a sample of the batch; and (b) validating the batch for distribution only if the number of moles of isoaspartate residues per mole of fusion protein is below a predetermined threshold number.

The invention also provides a process for validating a batch of a stable liquid pharmaceutical composition comprising an albumin-human growth hormone (hGH) fusion protein whose amino acid sequence is set forth as SEQ ID NO: 1 for distribution, the process comprising (a) determining the percentage of fusion protein having an isoaspartate residue at at least one of amino acid position D715, N734, N684, D692, or D697 in a sample of the batch; and (b) validating the batch for distribution only if the percentage of fusion protein having an isoaspartate residue at the one or more amino acid positions is below a threshold percentage predetermined for the at least one of amino acid positions D715, N734, N684, D692, or D697.

The invention also provides a method of treating a human patient in need of growth hormone therapy by periodically administering to the human patient for more than two weeks an effective amount of the stable liquid pharmaceutical composition of the invention.

The invention also provides a process for producing an albumin-human growth hormone (hGH) fusion protein whose amino acid sequence is set forth as SEQ ID NO: 1 comprising culturing a recombinant cell capable of expressing the fusion protein in a culture medium comprising a polysorbate, and isolating the fusion protein from the culture medium.

The invention also provides a process of producing a stable liquid pharmaceutical composition comprising 100 mg/mL of an albumin-human growth hormone (hGH) fusion protein whose amino acid sequence is set forth as SEQ ID NO: 1, comprising (a) forming a solution comprising the isolated fusion protein produced by any one of claims 83-92 and a liquid pharmaceutical acceptable excipient, and (b) concentrating the solution until the concentration of the fusion protein in the solution is 100 mg/mL if the concentration of fusion protein in the solution is less than 100 after step a), thereby providing a stable liquid pharmaceutical composition comprising 100 of the fusion protein.

### BRIEF DESCRIPTION OF THE DRAWINGS

FIG. 1: The amino acid sequence (SEQ ID NO: 1) of the albumin-hGH fusion protein TV-1106 (ALBUTROPIN™).

FIG. 2: Isoaspartate levels of TV-1106 formulations (PMT, HN, HNMT) at 2-8° C.

FIG. 3: Charge heterogeneity of TV-1106 formulations (PMT, HN, HNMT) at 2-8° C.

FIG. 4: P-1 charge variant of TV-1106 formulations (PMT, HN, HNMT) at 2-8° C.

FIG. 5: Purity measured by SE-HPLC of TV-1106 formulations (PMT, HN, HNMT) at 2-8° C.

FIG. 6: Purity measure by RP-HPLC of TV-1106 formulations (PMT, HN, HNMT) at 2-8° C.

FIG. 7: Relative potency of TV-1106 formulations (PMT, HN, HNMT) at 2-8° C.

FIG. 8: Isoaspartate level of TV-1106 formulations at pH 5.7, 6.0, and 6.3 at 2-8° C.

FIG. 9: Charge heterogeneity of TV-1106 formulations at pH 5.7, 6.0, and 6.3 at 2-8° C.

FIG. 10: P-1 charge variant of TV-1106 formulations at pH 5.7, 6.0, and 6.3 at 2-8° C.

FIG. 11: Purity measured by SE-HPLC of TV-1106 formulations at pH 5.7, 6.0, and 6.3 at 2-8° C.

FIG. 12: Purity measured by RP-HPLC of TV-1106 formulations at pH 5.7, 6.0, and 6.3 at 2-8° C.

FIG. 13: Relative potency of TV-1106 formulations at pH 5.7, 6.0, and 6.3 at 2-8° C.

FIG. 14: Projection of TV-1106 isoaspartate levels at the end of 24 and 36 months from 18 data.

FIG. 15: D715 isomerization (%) versus D692 or 697 isomerization (%).

FIG. 16: Susceptible site N149/734 deamidation (%) versus less susceptible site N99/684 deamidation (%).

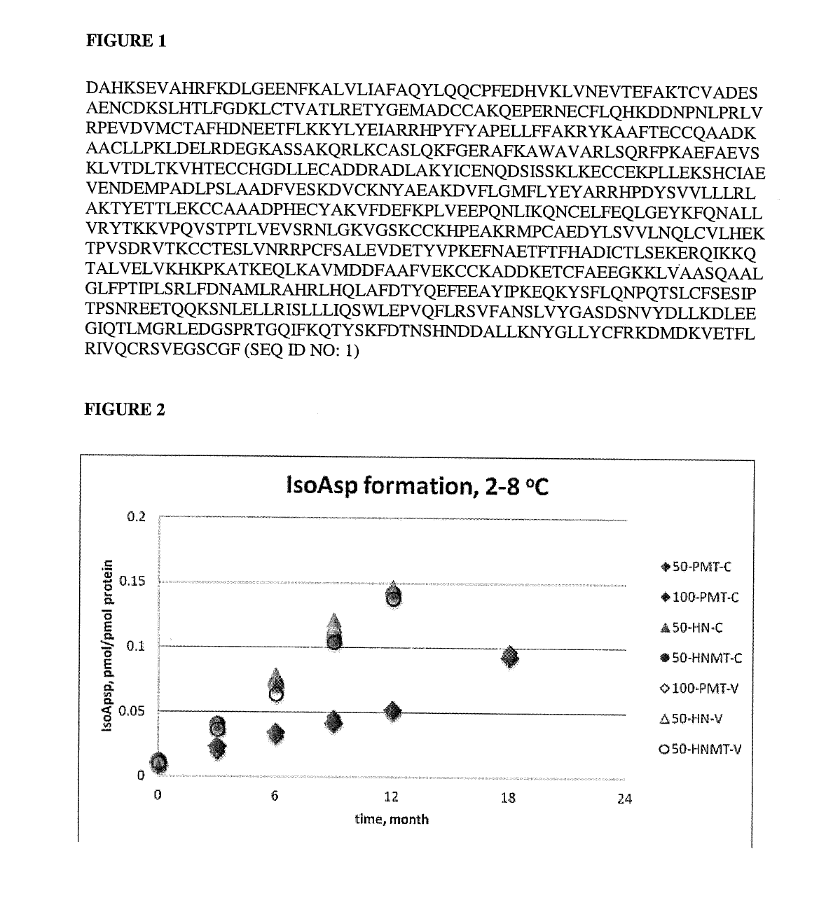

### DETAILED DESCRIPTION OF THE INVENTION

The invention provides a stable liquid pharmaceutical composition comprising: an albumin-human growth hormone (hGH) fusion protein whose amino acid sequence is set forth as SEQ ID NO: 1 a buffer, wherein the stable liquid pharmaceutical composition has a pH range of 5.5-6.5.

In some embodiments, 0.01-0.15 moles of isoaspartate residues are present per mole of fusion protein upon storage of the stable liquid pharmaceutical composition for 12 months at 2-8° C.

In some embodiments, less than 0.10, less than 0.09, less than 0.08, less than 0.07, less than 0.06, or less than 0.05 moles of isoaspartate residues are present per mole of fusion protein upon storage of the stable liquid pharmaceutical composition for 12 months at 2-8° C.

In some embodiments, less than 0.10 moles of isoaspartate residues are present per mole of fusion protein upon storage of the stable liquid pharmaceutical composition for 12 months at 2-8° C. In some embodiments, less than 0.09 moles of isoaspartate residues are present per mole of fusion protein upon storage of the stable liquid pharmaceutical composition for 12 months at 2-8° C. In some embodiments, less than 0.08 moles of isoaspartate residues are present per mole of fusion protein upon storage of the stable liquid pharmaceutical composition for 12 months at 2-8° C. In some embodiments, less than 0.07 moles of isoaspartate residues are present per mole of fusion protein upon storage of the stable liquid pharmaceutical composition for 12 months at 2-8° C. In some embodiments, less than 0.06 moles of isoaspartate residues are present per mole of fusion protein upon storage of the stable liquid pharmaceutical composition for 12 months at 2-8° C. In some embodiments, less than 0.05 moles of isoaspartate residues are present per mole of fusion protein upon storage of the stable liquid pharmaceutical composition for 12 months at 2-8° C.

In some embodiments, less than 0.25, less than 0.20, less than 0.15, or less than 0.10 moles of isoaspartate residues are present per mole of fusion protein upon storage of the stable liquid pharmaceutical composition for 24 months at 2-8° C.

In some embodiments, less than 0.25 moles of isoaspartate residues are present per mole of fusion protein upon storage of the stable liquid pharmaceutical composition for 24 months at 2-8° C. In some embodiments, less than 0.20 moles of isoaspartate residues are present per mole of fusion protein upon storage of the stable liquid pharmaceutical composition for 24 months at 2-8° C. In some embodiments, less than 0.15 moles of isoaspartate residues are present per mole of fusion protein upon storage of the stable liquid pharmaceutical composition for 24 months at 2-8° C. In some embodiments, less than 0.10 moles of isoaspartate residues are present per mole of fusion protein upon storage of the stable liquid pharmaceutical composition for 24 months at 2-8° C.

In some embodiments, less than 0.25, less than 0.20, or less than 0.15 moles of isoaspartate residues are present per mole of fusion protein upon storage of the stable liquid pharmaceutical composition for 36 months at 2-8° C.

In some embodiments, less than 0.25 moles of isoaspartate residues are present per mole of fusion protein upon storage of the stable liquid pharmaceutical composition for 36 months at 2-8° C. In some embodiments, less than 0.20 moles of isoaspartate residues are present per mole of fusion protein upon storage of the stable liquid pharmaceutical composition for 36 months at 2-8° C. In some embodiments, less than 0.15 moles of isoaspartate residues are present per mole of fusion protein upon storage of the stable liquid pharmaceutical composition for 36 months at 2-8° C.

In some embodiments, 1-5% of the fusion protein has an isoaspartate residue at amino acid position D715 upon storage of the stable liquid pharmaceutical composition for 6 months at 2-8° C.

In some embodiments, that less than 5%, less than 4%, or less than 3% of the fusion protein has an isoaspartate residue at amino acid position D715 upon storage of the stable liquid pharmaceutical composition for 6 months at 2-8° C.

In some embodiments, less than 5% of the fusion protein has an isoaspartate residue at amino acid position D715 upon storage of the stable liquid pharmaceutical composition for 6 months at 2-8° C. In some embodiments, less than 4% of the fusion protein has an isoaspartate residue at amino acid position D715 upon storage of the stable liquid pharmaceutical composition for 6 at 2-8° C. In some embodiments, less than 3% of the fusion protein has an isoaspartate residue at amino acid position D715 upon storage of the stable liquid pharmaceutical composition for 6 months at 2-8° C.

In some embodiments, less than 45% of the fusion protein has an isoaspartate residue at amino acid position D715 upon storage of the stable liquid pharmaceutical composition for 6 months at 25° C.

In some embodiments, 1-5% of the fusion protein has an isoaspartate residue at amino acid position N734 upon storage of the stable liquid pharmaceutical composition for 6 months at 2-8° C.

In some embodiments, less than 5%, less than 4%, or less than 3% of the fusion protein has an isoaspartate residue at amino acid position N734 upon storage of the stable liquid pharmaceutical composition for 6 months at 2-8° C.

In some embodiments, less than 5% of the fusion protein has an isoaspartate residue at amino acid position N734 upon storage of the stable liquid pharmaceutical composition for 6 months at 2-8° C. In some embodiments, less than 4% of the fusion protein has an isoaspartate residue at amino acid position N734 upon storage of the stable liquid pharmaceutical composition for 6 months at 2-8° C. In some embodiments, less than 3% of the fusion protein has an isoaspartate residue at amino acid position N734 upon storage of the stable liquid pharmaceutical composition for 6 months at 2-8° C.

In some embodiments, less than 45% of the fusion protein has an isoaspartate residue at amino acid position N734 upon storage of the stable liquid pharmaceutical composition for 6 months at 25° C.

In some embodiments, 0.1-1% of the fusion protein has an isoaspartate residue at amino acid position D692, amino acid position D697, or amino acid positions D692 and D697 upon storage of the stable liquid pharmaceutical composition for 6 months at 2-8° C.

In some embodiments, less than 1%, less than 0.9%, less than 0.8%, less than 0.7%, less than 0.6%, or less than 0.5% of the fusion protein has an isoaspartate residue at amino acid position D692, amino acid position D697, or amino acid positions D692 and D697 upon storage of the stable liquid pharmaceutical composition for 6 months at 2-8° C.

In some embodiments, less than 1% of the fusion protein has an isoaspartate residue at amino acid position D692, amino acid position D697, or amino acid positions D692 and D697 upon storage of the stable liquid pharmaceutical composition for 6 months at 2-8° C. In some embodiments, less than 0.9% of the fusion protein has an isoaspartate residue at amino acid position D692, amino acid position D697, or amino acid positions D692 and D697 upon storage of the stable liquid pharmaceutical composition for 6 months at 2-8° C. In some embodiments, less than 0.8% of the fusion protein has an isoaspartate residue at amino acid position D692, amino acid position D697, or amino acid positions D692 and D697 upon storage of the stable liquid pharmaceutical composition for 6 months at 2-8° C. In some embodiments, less than 0.7% of the fusion protein has an isoaspartate residue at amino acid position D692, amino acid position D697, or amino acid positions D692 and D697 upon storage of the stable liquid pharmaceutical composition for 6 at 2-8° C. In some embodiments, less than 0.6% of the fusion protein has an isoaspartate residue at amino acid position D692, amino acid position D697, or amino acid positions D692 and D697 upon storage of the stable liquid pharmaceutical composition for 6 months at 2-8° C. In some embodiments, less than 0.5% of the fusion protein has an isoaspartate residue at amino acid position D692, amino acid position D697, or amino acid positions D692 and D697 upon storage of the stable liquid pharmaceutical composition for 6 months at 2-8° C.

In some embodiments, less than 10%, less than 9%, or less than 8% of the fusion protein has an isoaspartate residue at amino acid position D692, amino acid position D697, or amino acid positions D692 and D697 upon storage of the stable liquid pharmaceutical composition for 6 at 25° C.

In some embodiments, less than 10% of the fusion protein has an isoaspartate residue at amino acid position D692, amino acid position D697, or amino acid positions D692 and D697 upon storage of the stable liquid pharmaceutical composition for 6 months at 25° C. In some embodiments, less than 9% of the fusion protein has an isoaspartate residue at amino acid position D692, amino acid position D697, or amino acid positions D692 and D697 upon storage of the stable liquid pharmaceutical composition for 6 months at 25° C. In some embodiments, less than 8% of the fusion protein has an isoaspartate residue at amino acid position D692, amino acid position D697, or amino acid positions D692 and D697 upon storage of the stable liquid pharmaceutical composition for 6 months at 25° C.

In some embodiments, 0.1-0.4% of the fusion protein has an isoaspartate residue at amino acid position N684 upon storage of the stable liquid pharmaceutical composition for 6 months at 2-8° C.

In some embodiments, less than 0.4%, less than 0.3%, or less than 0.25% of the fusion protein has an isoaspartate residue at amino acid position N684 upon storage of the stable liquid pharmaceutical composition for 6 months at 2-8° C.

In some embodiments, less than 0.4% of the fusion protein has an isoaspartate residue at amino acid position N684 upon storage of the stable liquid pharmaceutical composition for 6 months at 2-8° C. In some embodiments, less than 0.3% of the fusion protein has an isoaspartate residue at amino acid position N684 upon storage of the stable liquid pharmaceutical composition for 6 at 2-8° C. In some embodiments, less than 0.25% of the fusion protein has an isoaspartate residue at amino acid position N684 upon storage of the stable liquid pharmaceutical composition for 6 months at 2-8° C.

In some embodiments, less than 2.0%, less than 1.75%, or less than 1.5% of the fusion protein has an isoaspartate residue at amino acid position N684 upon storage of the stable liquid pharmaceutical composition for 6 months at 25° C.

In some embodiments, less than 2.0% of the fusion protein has an isoaspartate residue at amino acid position N684 upon storage of the stable liquid pharmaceutical composition for 6 months at 25° C. In some embodiments, less than 1.75% of the fusion protein has an isoaspartate residue at amino acid position N684 upon storage of the stable liquid pharmaceutical composition for 6 at 25° C. In some embodiments, less than 1.5% of the fusion protein has an isoaspartate residue at amino acid position N684 upon storage of the stable liquid pharmaceutical composition for 6 months at 25° C.

In some embodiments, the rate of formation of isoaspartate residues in the fusion protein at 2-8° C. is less than 0.006, less than 0.004, less than 0.003, or less than 0.00275 moles of isoaspartate residues per mole of fusion protein per month.

In some embodiments, the rate of formation of isoaspartate residues in the fusion protein at 2-8° C. is less than 0.006 moles of isoaspartate residues per mole of fusion protein per month. In some embodiments, the rate of formation of isoaspartate residues in the fusion protein at 2-8° C. is less than 0.004 moles of isoaspartate residues per mole of fusion protein per month. In some embodiments, the rate of formation of isoaspartate residues in the fusion protein at 2-8° C. is less than 0.003 moles of isoaspartate residues per mole of fusion protein per month. In some embodiments, the rate of formation of isoaspartate residues in the fusion protein at 2-8° C. is less than 0.00275 moles of isoaspartate residues per mole of fusion protein per month.

The invention also provides a stable liquid pharmaceutical composition comprising (i) an albumin-human growth hormone (hGH) fusion protein whose amino acid sequence is set forth as SEQ ID NO: 1, and (ii) a pharmaceutically acceptable excipient comprising a buffer, in which 0.01-0.15 moles of isoaspartate residues are present per mole of fusion protein after storage for 12 months at 2-8° C.

In some embodiments, less than 0.10, less than 0.09, less than 0.08, less than 0.07, less than 0.06, or less than 0.05 moles of isoaspartate residues are present per mole of fusion protein after storage for 12 months at 2-8° C.

In some embodiments, less than 0.10 moles of isoaspartate residues are present per mole of fusion protein after storage for 12 months at 2-8° C. In some embodiments, less than 0.09 moles of isoaspartate residues are present per mole of fusion protein after storage for 12 months at 2-8° C. In some embodiments, less than 0.08 moles of isoaspartate residues are present per mole of fusion protein after storage for 12 months at 2-8° C. In some embodiments, less than 0.07 moles of isoaspartate residues are present per mole of fusion protein after storage for 12 months at 2-8° C. In some embodiments, less than 0.06 moles of isoaspartate residues are present per mole of fusion protein after storage for 12 months at 2-8° C. In some embodiments, less than 0.05 moles of isoaspartate residues are present per mole of fusion protein after storage for 12 months at 2-8° C. In some embodiments, less than 0.25, less than 0.20, less than 0.15, or less than 0.10 moles of isoaspartate residues are present per mole of fusion protein after storage for 24 months at 2-8° C.

In some embodiments, less than 0.25 moles of isoaspartate residues are present per mole of fusion protein after storage for 24 months at 2-8° C. In some embodiments, less than 0.20 moles of isoaspartate residues are present per mole of fusion protein after storage for 24 months at 2-8° C. In some embodiments, less than 0.15 moles of isoaspartate residues are present per mole of fusion protein after storage for 24 months at 2-8° C. In some embodiments, less than 0.10 moles of isoaspartate residues are present per mole of fusion protein after storage for 24 months at 2-8° C.

In some embodiments, less than 0.25, less than 0.20, or less than 0.15 moles of isoaspartate residues are present per mole of fusion protein after storage for 36 months at 2-8° C.

In some embodiments, less than 0.25 moles of isoaspartate residues are present per mole of fusion protein after storage for 36 months at 2-8° C. In some embodiments, less than 0.20 moles of isoaspartate residues are present per mole of fusion protein after storage for 36 months at 2-8° C. In some embodiments, less than 0.15 moles of isoaspartate residues are present per mole of fusion protein after storage for 36 months at 2-8° C.

The invention also provides a stable liquid pharmaceutical composition comprising (i) an albumin-human growth hormone (hGH) fusion protein whose amino acid sequence is set forth as SEQ ID NO: 1, and (ii) a pharmaceutically acceptable excipient comprising a buffer, in which 1-5% of the fusion protein has an isoaspartate residue at amino acid position D715 after storage for 6 months at 2-8° C.

In some embodiments, less than 5%, less than 4%, or less than 3% of the fusion protein has an isoaspartate residue at amino acid position D715 after storage for 6 months at 2-8° C.

In some embodiments, less than 5% of the fusion protein has an isoaspartate residue at amino acid position D715 after storage for 6 months at 2-8° C. In some embodiments, less than 4% of the fusion protein has an isoaspartate residue at amino acid position D715 after storage for 6 months at 2-8° C. In some embodiments, less than 3% of the fusion protein has an isoaspartate residue at amino acid position D715 after storage for 6 months at 2-8° C.

In some embodiments, 1-5% of the fusion protein has an isoaspartate residue at amino acid position N734 after storage for 6 months at 2-8° C.

In some embodiments, less than 5%, less than 4%, or less than 3% of the fusion protein has an isoaspartate residue at amino acid position N734 of SEQ ID NO:1 after storage for 6 months at 2-8° C.

In some embodiments, less than 5% of the fusion protein has an isoaspartate residue at amino acid position N734 of SEQ ID NO:1 after storage for 6 months at 2-8° C. In some embodiments, less than 4% of the fusion protein has an isoaspartate residue at amino acid position N734 of SEQ ID NO: 1 after storage for 6 months at 2-8° C. In some embodiments, less than 3% of the fusion protein has an isoaspartate residue at amino acid position N734 of SEQ ID NO: 1 after storage for 6 months at 2-8° C.

In some embodiments, 0.1-1% of the fusion protein has an isoaspartate residue at amino acid position D692, amino acid position D697, or amino acid positions D692 and D697 after storage for 6 months at 2-8° C.

In some embodiments, less than 1%, less than 0.9%, less than 0.8%, less than 0.7%, less than 0.6%, or less than 0.5% of the fusion protein has an isoaspartate residue at amino acid position D692, amino acid position D697, or amino acid positions D692 and D697 after storage for 6 months at 2-8° C.

In some embodiments, less than 1% of the fusion protein has an isoaspartate residue at amino acid position D692, amino acid position D697, or amino acid positions D692 and D697 after storage for 6 months at 2-8° C. In some embodiments, less than 0.9% of the fusion protein has an isoaspartate residue at amino acid position D692, amino acid position D697, or amino acid positions D692 and D697 after storage for 6 months at 2-8° C. In some embodiments, less than 0.8% of the fusion protein has an isoaspartate residue at amino acid position D692, amino acid position D697, or amino acid positions D692 and D697 after storage for 6 months at 2-8° C. In some embodiments, less than 0.7% of the fusion protein has an isoaspartate residue at amino acid position D692, amino acid position D697, or amino acid positions D692 and D697 after storage for 6 months at 2-8° C. In some embodiments, less than 0.6% of the fusion protein has an isoaspartate residue at amino acid position D692, amino acid position D697, or amino acid positions D692 and D697 after storage for 6 months at 2-8° C. In some embodiments, less than 0.5% of the fusion protein has an isoaspartate residue at amino acid position D692, amino acid position D697, or amino acid positions D692 and D697 after storage for 6 months at 2-8° C.

In some embodiments, 0.1-0.4% of the fusion protein has an isoaspartate residue at amino acid position N684 upon storage of the stable liquid pharmaceutical composition for 6 months at 2-8° C.

In some embodiments, less than 0.4%, less than 0.3%, or less than 0.25% of the fusion protein has an isoaspartate residue at amino acid position N684 upon storage of the stable liquid pharmaceutical composition for 6 months at 2-8° C.

In some embodiments, less than 0.4% of the fusion protein has an isoaspartate residue at amino acid position N684 upon storage of the stable liquid pharmaceutical composition for 6 months at 2-8° C. In some embodiments, less than 0.3% of the fusion protein has an isoaspartate residue at amino acid position N684 upon storage of the stable liquid pharmaceutical composition for 6 at 2-8° C. In some embodiments, less than 0.25% of the fusion protein has an isoaspartate residue at amino acid position N684 upon storage of the stable liquid pharmaceutical composition for 6 months at 2-8° C.

In some embodiments, 1-5% of the fusion protein has an isoaspartate residue at amino acid position D715 after storage for 6 months at 2-8° C., 1-5% of the fusion protein has an isoaspartate residue at amino acid position N734 after storage for 6 months at 2-8° C., 0.1-1% of the fusion protein has an isoaspartate residue at amino acid positions D692 and D697 after storage for 6 at 2-8° C., and 0.1-0.4% of the fusion protein has an isoaspartate residue at amino acid position N684 upon storage of the stable liquid pharmaceutical composition for 6 months at 2-8° C.

In some embodiments, 3% of the fusion protein has an isoaspartate residue at amino acid position D715 after storage for 6 months at 2-8° C., 3% of the fusion protein has an isoaspartate residue at amino acid position N734 after storage for 6 months at 2-8° C., 0.6% of the fusion protein has an isoaspartate residue at amino acid positions D692 and D697 after storage for 6 months at 2-8° C., and 0.2% of the fusion protein has an isoaspartate residue at amino acid position N684 upon storage of the stable liquid pharmaceutical composition for 6 months at 2-8° C.

In some embodiments, the buffer comprises phosphate. In some embodiments, the buffer comprises sodium phosphate.

In some embodiments, the stable liquid pharmaceutical composition comprises 10-50 mM phosphate.

In some embodiments, the stable liquid pharmaceutical composition comprises 20 mM phosphate. In some embodiments, the buffer comprises histidine.

In some embodiments, the stable liquid pharmaceutical composition comprises 10-50 mM histidine.

In some embodiments, the stable liquid pharmaceutical composition comprises 20 mM histidine.

In some embodiments, the stable liquid pharmaceutical composition further comprises mannitol.

In some embodiments, the stable liquid pharmaceutical composition comprises 25-250 mM mannitol.

In some embodiments, the stable liquid pharmaceutical composition comprises 180 mM mannitol.

In some embodiments, the stable liquid pharmaceutical composition further comprises trehalose.

In some embodiments, the stable liquid pharmaceutical composition comprises 25-250 mM trehalose.

In some embodiments, the stable liquid pharmaceutical composition comprises 55-75 mM trehalose.

In some embodiments, the stable liquid pharmaceutical composition comprises 60 mM trehalose.

In some embodiments, the stable liquid pharmaceutical composition comprise 75 mM trehalose.

In some embodiments, the stable liquid pharmaceutical composition has an osmolality from 250 to 350 mOsm/kg.

In some embodiments, the pH of the stable liquid pharmaceutical composition is 5.7-6.3.

In some embodiments, the pH of the stable liquid pharmaceutical composition is 6.0.

In some embodiments, the stable liquid pharmaceutical composition comprises 20 mM sodium phosphate, 180 mM mannitol, 60 mM trehalose, pH 6.0.

In some embodiments, the stable liquid pharmaceutical composition further comprises sodium chloride.

In some embodiments, the stable liquid pharmaceutical composition comprises 25-500 mM sodium chloride.

In some embodiments, the concentration of the fusion protein in the above-described stable liquid pharmaceutical compositions is 2-200 mg/mL. In some embodiments, the concentration of the fusion protein is 100 mg/mL.

In some embodiments, the stable liquid pharmaceutical compositions further comprise polysorbate 80.

In some embodiments, the stable liquid pharmaceutical compositions comprise 1.2 mg of polysorbate 80 per 100 mg of the fusion protein.

In some embodiments, the stable liquid pharmaceutical composition comprises 100 mg/mL TV-1106, 20 mM sodium phosphate, 180 mM mannitol, 60 mM trehalose, 1.2 mg polysorbate 80 per 100 mg of fusion protein, pH 6.0.

In some embodiments of the above-described stable liquid pharmaceutical compositions, the purity of the fusion protein decreases by 2.5% or less after incubation at 25° C. for 72 hours.

In some embodiments of the above-described stable liquid pharmaceutical compositions, the presence of isoaspartate residues is determined by (i) an assay which detects isoaspartate residues via protein isoaspartyl methyltransferase (PIMT)-catalyzed generation of S-adenosyl homocysteine (SAH), (ii) isoelectric focusing, (iii) ion-exchange chromatography, or (iv) protein mapping and mass spectrometry.

In some embodiments, the presence of isoaspartate residues is determined by an assay which detects isoaspartate residues via protein isoaspartyl methyltransferase (PIMT)-catalyzed generation of S-adenosyl homocysteine (SAH). An example of such an assay is the ISOQUANT® assay (Promega). In some embodiments, the presence of isoaspartate residues is determined by isoelectric focusing. In some embodiments, the presence of isoaspartate residues is determined by ion-exchange chromatography. In some embodiments, the presence of isoaspartate residues is determined by protein mapping and mass spectrometry.

The invention provides a package comprising any of the stable liquid pharmaceutical compositions of the invention and a container.

In some embodiments, the container is a cartridge, a vial, a pre-filled syringe, an infusion pump, or an injection pen. In some embodiments, the container is a cartridge. In some embodiments, the container is a vial. In some embodiments, the container is a pre-filled syringe. In some embodiments, the container is an infusion pump. In some embodiments, the container is an injection pen.

In some embodiments, the package further comprises silicone oil.

In some embodiments, the package comprises 0.2 mg to 200 mg of the fusion protein.

In some embodiments, the package comprises 0.2 mg, 0.4 mg, 0.6 mg, 0.8 mg, 1.0 mg, 1.2 mg, 1.4 mg, 1.6 mg, 1.8 mg, 2.0 mg, 4 mg, 5 mg, 6 mg, 8 mg, 8.8 mg, 10 mg, 12 mg, 15 mg, 20 mg, 24 mg, 25 mg, 30 mg, 35 mg, 40 mg, 45 mg, 50 mg, 60 mg, 70 mg, 80 mg, 90 mg, 100 mg, 150 or 200 mg of the fusion protein.

In some embodiments, the package comprises 0.2 to 2 mL of the stable liquid pharmaceutical composition.

In some embodiments, the package comprises 1.5 mL of the stable liquid pharmaceutical composition. In some embodiments, the package comprises 1.5 mL of a table liquid pharmaceutical composition of the invention having 100 mg/mL of the fusion protein.

The invention also provides a process for preparing a stable liquid pharmaceutical composition comprising an albumin-human growth hormone (hGH) fusion protein whose amino acid sequence is set forth as SEQ ID NO: 1, the process comprising (a) determining the number of moles of isoaspartate residues present per mole of fusion protein in a batch of the fusion protein; and (b) preparing the stable liquid pharmaceutical composition from the batch only if less than 0.05, less than 0.04, less than 0.03, or less than 0.02 moles of isoaspartate residues are present per mole of fusion protein in the batch.

In some embodiments, step (b) comprises preparing the stable liquid pharmaceutical composition from the batch only if less than 0.05 moles of isoaspartate residues are present per mole of fusion protein in the batch. In some embodiments, step (b) comprises preparing the stable liquid pharmaceutical composition from the batch only if less than 0.04 moles of isoaspartate residues are present per mole of fusion protein in the batch. In some embodiments, step (b) comprises preparing the stable liquid pharmaceutical composition from the batch only if less than 0.03 moles of isoaspartate residues are present per mole of fusion protein in the batch. In some embodiments, step (b) comprises preparing the stable liquid pharmaceutical composition from the batch only if less than 0.02 moles of isoaspartate residues are present per mole of fusion protein in the batch.

The invention also provides a process for preparing a stable liquid pharmaceutical composition comprising an albumin-human growth hormone (hGH) fusion protein whose amino acid sequence is set forth as SEQ ID NO: 1, the process comprising (a) determining the percentage of fusion protein having an isoaspartate residue at at least one of amino acid positions D715, N734, N684, D692, or D697 in a batch of fusion protein; and (b) preparing the pharmaceutical composition from the batch only if the percentage of fusion protein having an isoaspartate residue at the one or more amino acid positions determined in step (a) is below a threshold percentage predetermined for the at least one of amino acid positions D715, N734, N684, D692, or D697.

In some embodiments of the process just described, step (a) comprises determining the percentage of fusion protein having an isoaspartate residue at only one of amino acid positions D715, N734, N684, D692, or D697. In some embodiments, step (a) comprises determining the percentage of fusion protein having an isoaspartate residue at any combination of two of amino acid positions D715, N734, N684, D692, or D697. In some embodiments, step (a) comprises determining the percentage of fusion protein having an isoaspartate residue at any combination of three of amino acid positions D715, N734, N684, D692, or D697. In some embodiments, step (a) comprises determining the percentage of fusion protein having an isoaspartate residue at any combination of four of amino acid positions D715, N734, N684, D692, or D697. In some embodiments, step (a) comprises determining the percentage of fusion protein having an isoaspartate residue at amino acid positions D715, N734, N684, D692, and D697.

In some embodiments, step (a) consists of determining the percentage of fusion protein having an isoaspartate residue at any combination of two of amino acid positions D715, N734, N684, D692, or D697. In some embodiments, step (a) consists of determining the percentage of fusion protein having an isoaspartate residue at any combination of three of amino acid positions D715, N734, N684, D692, or D697. In some embodiments, step (a) consists of determining the percentage of fusion protein having an isoaspartate residue at any combination of four of amino acid positions D715, N734, N684, D692, or D697. In some embodiments, step (a) consists of determining the percentage of fusion protein having an isoaspartate residue at amino acid positions D715, N734, N684, D692, and D697.

In some embodiments, the threshold percentage is (i) 5%, 4%, 3%, 2%, or 1% for amino acid position D715, (ii) 5%, 4%, 3%, 2%, or 1% for amino acid position N734, (iii) 0.4%, 0.3%, or 0.25% for amino acid position N684, (iv) 0.7%, 0.6%, 0.5%, 0.4%, 0.3%, or 0.2% for amino acid position D692, and (v) 0.7%, 0.6%, 0.5%, 0.4%, 0.3%, or 0.2% for amino acid position D697.

In some embodiments, the threshold percentage is 5% for amino acid position D715. In some embodiments, the threshold percentage is 4% for amino acid position D715. In some embodiments, the threshold percentage is 3% for amino acid position D715. In some embodiments, the threshold percentage is 2% for amino acid position D715. In some embodiments, the threshold percentage is 1% for amino acid position D715. In some embodiments, the threshold percentage is 5% for amino acid position N734. In some embodiments, the threshold percentage is 4% for amino acid position N734. In some embodiments, the threshold percentage is 3% for amino acid position N734. In some embodiments, the threshold percentage is 2% for amino acid position N734. In some embodiments, the threshold percentage is 1% for amino acid position N734. In some embodiments, the threshold percentage is 0.4% for amino acid position N684. In some embodiments, the threshold percentage is 0.3% for amino acid position N684. In some embodiments, the threshold percentage is 0.25% for amino acid position N684. In some embodiments, the threshold percentage is 0.7% for amino acid position D692. In some embodiments, the threshold percentage is 0.6% for amino acid position D692. In some embodiments, the threshold percentage is 0.5% for amino acid position D692. In some embodiments, the threshold percentage is 0.4% for amino acid position D692. In some embodiments, the threshold percentage is 0.3% for amino acid position D692. In some embodiments, the threshold percentage is 0.2% for amino acid position D692. In some embodiments, the threshold percentage is 0.7% for amino acid position D697. In some embodiments, the threshold percentage is 0.6% for amino acid position D697. In some embodiments, the threshold percentage is 0.5% for amino acid position D697. In some embodiments, the threshold percentage is 0.4% for amino acid position D697. In some embodiments, the threshold percentage is 0.3% for amino acid position D697. In some embodiments, the threshold percentage is 0.2% for amino acid position D697.

The invention also provides a process for validating a batch of a stable liquid pharmaceutical composition comprising an albumin-human growth hormone (hGH) fusion protein whose amino acid sequence is set forth as SEQ ID NO: 1 for distribution, the process comprising (a) determining the number of moles of isoaspartate residues present per mole of fusion protein in a sample of the batch; and (b) validating the batch for distribution only if the number of moles of isoaspartate residues per mole of fusion protein is below a predetermined threshold number.

In some embodiments of the process just described, the predetermined threshold number is 0.20, 0.10, 0.05, 0.04, 0.03, or 0.02.

In some embodiments, the predetermined threshold number is 0.20. In some embodiments, the predetermined threshold number is 0.15. In some embodiments, the predetermined threshold number is 0.10. In some embodiments, the predetermined threshold number is 0.05. In some embodiments, the predetermined threshold number is 0.04. In some embodiments, the predetermined threshold number is 0.03. In some embodiments, the predetermined threshold number is 0.02.

In some embodiments, step (a) comprises determining the number of moles of isoaspartate residues present per mole of fusion protein in a sample of the batch after the batch is stored at conditions which replicate storage at 25° C. for 3 months.

In some embodiments, the predetermined threshold number is 0.40, 0.35, 0.30, or 0.25.

In some embodiments, the predetermined threshold number is 0.40. In some embodiments, the predetermined threshold number is 0.35. In some embodiments, the predetermined threshold number is 0.30. In some embodiments, the predetermined threshold number is 0.25.

The invention also provides a process for validating a batch of a stable liquid pharmaceutical composition comprising an albumin-human growth hormone (hGH) fusion protein whose amino acid sequence is set forth as SEQ ID NO: 1 for distribution, the process comprising (a) determining the percentage of fusion protein having an isoaspartate residue at at least one of amino acid position D715, N734, N684, D692, or D697 in a sample of the batch; and (b) validating the batch for distribution only if the percentage of fusion protein having an isoaspartate residue at the one or more amino acid positions is below a threshold percentage predetermined for the at least one of amino acid positions D715, N734, N684, D692, or D697.

In some embodiments of the invention just described, step (a) comprises determining the percentage of fusion protein having an isoaspartate residue at only one of amino acid positions D715, N734, N684, D692, or D697 in a sample of the batch. In some embodiments, step (a) comprises determining the percentage of fusion protein having an isoaspartate residue at any combination of two of amino acid positions D715, N734, N684, D692, or D697 in a sample of the batch. In some embodiments, step (a) comprises determining the percentage of fusion protein having an isoaspartate residue at any combination of three of amino acid positions D715, N734, N684, D692, or D697 in a sample of the batch. In some embodiments, step (a) comprises determining the percentage of fusion protein having an isoaspartate residue at any combination of four of amino acid positions D715, N734, N684, D692, or D697 in a sample of the batch. In some embodiments, step (a) comprises determining the percentage of fusion protein having an isoaspartate residue at amino acid positions D715, N734, N684, D692, and D697 in a sample of the batch.

In some embodiments, step (a) consists of determining the percentage of fusion protein having an isoaspartate residue at any combination of two of amino acid positions D715, N734, N684, D692, or D697 in a sample of the batch. In some embodiments, step (a) consists of determining the percentage of fusion protein having an isoaspartate residue at any combination of three of amino acid positions D715, N734, N684, D692, or D697 in a sample of the batch. In some embodiments, step (a) consists of determining the percentage of fusion protein having an isoaspartate residue at any combination of four of amino acid positions D715, N734, N684, D692, or D697 in a sample of the batch. In some embodiments, step (a) consists of determining the percentage of fusion protein having an isoaspartate residue at amino acid positions D715, N734, N684, D692, and D697 a sample of the batch.

In some embodiments, the threshold percentage is (i) 5%, 4%, 3%, 2%, or 1% for amino acid position D715, (ii) 5%, 4%, 3%, 2%, or 1% for amino acid position N734, (iii) 0.4%, 0.3%, or 0.25% for amino acid position N684, (iv) 0.7%, 0.6%, 0.5%, 0.4%, 0.3%, or 0.2% for amino acid position D692, and (v) 0.7%, 0.6%, 0.5%, 0.4%, 0.3%, or 0.2% for amino acid position D697.

In some embodiments, the threshold percentage is 5% for amino acid position D715. In some embodiments, the threshold percentage is 4% for amino acid position D715. In some embodiments, the threshold percentage is 3% for amino acid position D715. In some embodiments, the threshold percentage is 2% for amino acid position D715. In some embodiments, the threshold percentage is 1% for amino acid position D715. In some embodiments, the threshold percentage is 5% for amino acid position N734. In some embodiments, the threshold percentage is 4% for amino acid position N734. In some embodiments, the threshold percentage is 3% for amino acid position N734. In some embodiments, the threshold percentage is 2% for amino acid position N734. In some embodiments, the threshold percentage is 1% for amino acid position N734. In some embodiments, the threshold percentage is 0.4% for amino acid position N684. In some embodiments, the threshold percentage is 0.3% for amino acid position N684. In some embodiments, the threshold percentage is 0.25% for amino acid position N684. In some embodiments, the threshold percentage is 0.7% for amino acid position D692. In some embodiments, the threshold percentage is 0.6% for amino acid position D692. In some embodiments, the threshold percentage is 0.5% for amino acid position D692. In some embodiments, the threshold percentage is 0.4% for amino acid position D692. In some embodiments, the threshold percentage is 0.3% for amino acid position D692. In some embodiments, the threshold percentage is 0.2% for amino acid position D692. In some embodiments, the threshold percentage is 0.7% for amino acid position D697. In some embodiments, the threshold percentage is 0.6% for amino acid position D697. In some embodiments, the threshold percentage is 0.5% for amino acid position D697. In some embodiments, the threshold percentage is 0.4% for amino acid position D697. In some embodiments, the threshold percentage is 0.3% for amino acid position D697. In some embodiments, the threshold percentage is 0.2% for amino acid position D697.

In some embodiments, step (a) comprises determining the percentage of fusion protein having an isoaspartate at at least one of amino acid position D715, N734, N684, D692, or D697 in a sample of the batch after the batch is stored at conditions which replicate storage at 25° C. for 3 months.

In some embodiments, the threshold percentage is (i) 40%, 35%, 30%, 25%, or 20% for amino acid position D715, (ii) 40%, 35%, 30%, 25%, or 20% for amino acid position N734, (iii) 1.0%, or 0.9% for amino acid position N684, (iv) 10%, 9%, 8%, 7%, 6%, or 5% for amino acid position D692 and (v) 10%, 9%, 8%, 7%, 6%, or 5% for amino acid position D697.

In some embodiments, the threshold percentage is 40% for amino acid position D715. In some embodiments, the threshold percentage is 35% for amino acid position D715. In some embodiments, the threshold percentage is 30% for amino acid position D715. In some embodiments, the threshold percentage is 25% for amino acid position D715. In some embodiments, the threshold percentage is 20% for amino acid position D715. In some embodiments, the threshold percentage is 40% for amino acid position N734. In some embodiments, the threshold percentage is 35% for amino acid position N734. In some embodiments, the threshold percentage is 30% for amino acid position N734. In some embodiments, the threshold percentage is 25% for amino acid position N734. In some embodiments, the threshold percentage is 20% for amino acid position N734. In some embodiments, the threshold percentage is 1.0% for amino acid position N684. In some embodiments, the threshold percentage is 0.9% for amino acid position N684. In some embodiments, the threshold percentage is 10% for amino acid position D692. In some embodiments, the threshold percentage is 9% for amino acid position D692. In some embodiments, the threshold percentage is 8% for amino acid position D692. In some embodiments, the threshold percentage is 7% for amino acid position D692. In some embodiments, the threshold percentage is 6% for amino acid position D692. In some embodiments, the threshold percentage is 5% for amino acid position D692. In some embodiments, the threshold percentage is 10% for amino acid position D697. In some embodiments, the threshold percentage is 9% for amino acid position D697. In some embodiments, the threshold percentage is 8% for amino acid position D697. In some embodiments, the threshold percentage is 7% for amino acid position D697. In some embodiments, the threshold percentage is 6% for amino acid position D697. In some embodiments, the threshold percentage is 5% for amino acid position D692.

In some embodiments of the validation processes just described, the process further comprises after step (b), step (c) of distributing the batch; and step (d) of monitoring the amount of isoaspartate residues in the fusion protein after distributing the batch.

In some embodiments of the processes of the invention, the presence of isoaspartate residues is determined by (i) an assay which detects isoaspartate residues via protein isoaspartyl methyltransferase (PIMT)-catalyzed generation of S-adenosyl homocysteine (SAH), (ii) isoelectric focusing, (iii) ion-exchange chromatography, or (iv) protein mapping and mass spectrometry. In some embodiments, the presence of isoaspartate residues is determined by an assay which detects isoaspartate residues via protein isoaspartyl methyltransferase (PIMT)-catalyzed generation of S-adenosyl homocysteine (SAH). In some embodiments, the presence of isoaspartate residues is determined by isoelectric focusing. In some embodiments, the presence of isoaspartate residues is determined by ion-exchange chromatography. In some embodiments, the presence of isoaspartate residues is determined by protein mapping and mass spectrometry.

The invention also provides a method of treating a human patient in need of growth hormone therapy by periodically administering to the human patient for more than two weeks an effective amount of the stable liquid pharmaceutical composition of the invention.

In some embodiments, the patient suffers from at least one of the following: growth hormone deficiency, Ulrich-Turner Syndrome, Prader-Willi Syndrome, Idiopathic Short Stature, Shox Deficiency, born small for gestational age (SGA), or Renal Insufficiency.

In some embodiments, the method comprises periodically administering the stable liquid pharmaceutical composition to the human patient one to four times every two weeks for more than two weeks.

In some embodiments, the stable liquid pharmaceutical composition is administered once a week, twice per week, or once every two weeks. In some embodiments, the stable liquid pharmaceutical composition is administered once a week. In some embodiments, the stable liquid pharmaceutical composition is administered twice a week. In some embodiments, the stable liquid pharmaceutical composition is administered once every two weeks.

In some embodiments, the effective amount of the stable liquid pharmaceutical composition is an amount which provides 1 to 200 mg of the hGH fusion protein to the patient per week.

In some embodiments, the stable liquid pharmaceutical composition is administered by subcutaneous injection, intramuscular injection, or intravenous administration. In some embodiments, the stable liquid pharmaceutical composition is administered by subcutaneous injection. In some embodiments, the stable liquid pharmaceutical composition is administered by intramuscular injection. In some embodiments, the stable liquid pharmaceutical composition is administered by intravenous administration.

In some embodiments, the stable liquid pharmaceutical composition is administered by subcutaneous injection by an injection pen.

In some embodiments, the patient has growth hormone deficiency.

In some embodiments, the patient is prepubescent.

In some embodiments, the patient is administered an amount of the stable liquid pharmaceutical composition which provides 0.015 to 3 mg/kg/week, 0.554 mg/kg/week, 0.924 mg/kg/week, or 1.2 of the fusion protein.

In some embodiments, the patient is administered an amount of the stable liquid pharmaceutical composition which provides 0.554 mg/kg/week of the fusion protein. In some embodiments, the patient is administered an amount of the stable liquid pharmaceutical composition which provides 0.924 mg/kg/week of the fusion protein. In some embodiments, the patient is administered an amount of the stable liquid pharmaceutical composition which provides 1.2 mg/kg/week of the fusion protein.

In some embodiments, the patient has attained puberty.

In some embodiments, the patient is administered an amount of the stable liquid pharmaceutical composition which provides up to 100 mg of fusion protein per administration.

In some embodiments, the patient is administered an amount of the stable liquid pharmaceutical composition which provides up to 50 mg of fusion protein per administration.

In some embodiments, the effective amount of the stable liquid pharmaceutical composition is determined by a titration based on the IGF-I level in the human patient.

In some embodiments, the treating is keeping the IGF-I level of the patient in the human patient within a normal range.

The invention also provides a process for producing an albumin-human growth hormone (hGH) fusion protein whose amino acid sequence is set forth as SEQ ID NO: 1 comprising culturing a recombinant cell capable of expressing the fusion protein in a culture medium comprising a polysorbate, and isolating the fusion protein from the culture medium.

Cell culture media and culture conditions described in the art can be used in the process just described. Exemplary media and culture conditions are described in Therapeutic Proteins: Methods and Protocols, C. M. Smales, D. C. James, ed., Humana Press, 2005 (incorporated by reference in its entirety). In some embodiments, the culture medium comprises a carbon source, a nitrogen source, and minerals. In some embodiments, the culture medium comprises yeast extract, peptone, and glucose. In some embodiments, the culture medium comprises YPD broth. In some embodiments, the fusion protein is isolated from the culture medium using cation exchange chromatography, anion exchange chromatography, gel permeation chromatography, or a combination thereof.

In some embodiments, the polysorbate added to the culture medium before the culturing begins.

In some embodiments, the polysorbate is added to the culture medium after the culturing begins.

In some embodiments, the polysorbate is added to the culture medium at least 1 hour, at least 2 at least 4 hours, at least 8 hours, at least 16 hours, at least 1 day, at least 2 days, at least 3 at least 4 days, or at least 5 days before the fusion protein is isolated.

In some embodiments, the polysorbate is added to the culture medium at least 1 hour before the fusion protein is isolated. In some embodiments, the polysorbate is added to the culture medium at least 2 hours before the fusion protein is isolated. In some embodiments, the polysorbate is added to the culture medium at least 4 hours before the fusion protein is isolated. In some embodiments, the polysorbate is added to the culture medium at least 8 hours before the fusion protein is isolated. In some embodiments, the polysorbate is added to the culture medium at least 16 hours before the fusion protein is isolated. In some embodiments, the polysorbate is added to the culture medium at least 24 hours before the fusion protein is isolated. In some embodiments, the polysorbate is added to the culture medium at least 1 day before the fusion protein is isolated. In some embodiments, the polysorbate is added to the culture medium at least 2 days before the fusion protein is isolated. In some embodiments, the polysorbate is added to the culture medium at least 2 days before the fusion protein is isolated. In some embodiments, the polysorbate is added to the culture medium at least 4 days before the fusion protein is isolated. In some embodiments, the polysorbate is added to the culture medium at least 5 days before the fusion protein is isolated.

In some embodiments, the polysorbate is present in the culture medium at a concentration below its critical micelle concentration (CMC). In other embodiments, the polysorbate is present in the culture medium at its CMC. In other embodiments, the polysorbate is present in the culture medium at a concentration above its critical micelle concentration (CMC). In some embodiments, the concentration of the polysorbate is maintained for the duration of the culturing. In some embodiments, the culture medium comprises 0.5 to 10 mL/L of polysorbate.

In some embodiments, the culture is a batch culture or a fed-batch culture.

In some embodiments, the culture is a continuous culture.

In some embodiments, the recombinant cell is a yeast cell. In some embodiments, the recombinant cell is a yeast cell which has been transformed with an expression vector coding for the fusion protein.

In some embodiments, the recombinant cell is a S. cerevisiae strain.

In some embodiments, the polysorbate is polysorbate 80. In some embodiments, the culture medium comprises 0.9 mL/L, 4.5 mL/L, or 9 mL/L of polysorbate 80.

In some embodiments, the volume of the culture medium is greater than 200 L.

In some embodiments, the volume of the culture medium is at least 500 L, at least 1,000 L, or at least 1,500 L.

In some embodiments, the volume of the culture medium is at least 500 L. In some embodiments, the volume of the culture medium is at least 1,000 L. In some embodiments, the volume of the culture medium is at least 1,500 L.

The invention also provides a process of producing a stable liquid pharmaceutical composition comprising 100 mg/mL of an albumin-human growth hormone (hGH) fusion protein whose amino acid sequence is set forth as SEQ ID NO: 1, comprising (a) forming a solution comprising the isolated fusion protein produced by any one of claims 83-92 and a liquid pharmaceutically acceptable excipient, (b) concentrating the solution until the concentration of the fusion protein in the solution is 100 mg/mL only if the concentration of fusion protein in the solution is less than 100 mg/mL after step a), and (c) diluting the solution until the concentration of the fusion protein in the sample is 100 mg/mL only if the concentration of the fusion protein in the solution is more than 100 mg/mL after step a), thereby providing a stable liquid pharmaceutical composition comprising 100 mg/mL of the fusion protein.

In some embodiments of the process just described, step (b) and step (c) are omitted.

In some embodiments of the process, forming the solution comprises reconstituting lyophilized isolated fusion protein with an amount of the liquid pharmaceutically acceptable excipient, adding a liquid comprising the isolated fusion protein to an amount of the liquid pharmaceutically acceptable excipient, dialyzing a liquid comprising the isolated fusion protein against an amount of the liquid pharmaceutically acceptable excipient, diafiltering a liquid comprising the isolated fusion protein against an amount of the liquid pharmaceutically acceptable excipient or a combination thereof.

In some embodiments of the process, forming the solution comprises reconstituting lyophilized isolated fusion protein with an amount of the liquid pharmaceutically acceptable excipient. In some embodiments of the process, forming the solution comprises adding a liquid comprising the isolated fusion protein to an amount of the liquid pharmaceutically acceptable excipient. In some embodiments of the process, forming the solution comprises dialyzing a liquid comprising the isolated fusion protein against an amount of the liquid pharmaceutically acceptable excipient. In some embodiments of the process, forming the solution comprises diafiltering a liquid comprising the isolated fusion protein against an amount of the liquid pharmaceutically acceptable excipient or a combination thereof.

In some embodiments, solution is formed after thawing a liquid comprising the isolated fusion protein.

In the some embodiments, the pharmaceutically acceptable excipient comprises 20 mM sodium phosphate, 180 mM mannitol, 60 mM trehalose, pH 6.0.

In some embodiments of the process, the stable liquid pharmaceutical composition comprises a buffer, mannitol, and trehalose, and wherein the liquid pharmaceutical formulation has a pH of 5.5-6.5.

In some embodiments of the process, the stable liquid pharmaceutical composition comprises 20 phosphate, 180 mM mannitol, 60 mM trehalose, pH 6.0.

In some embodiments of the process, the stable liquid pharmaceutical composition comprises 1.2 of polysorbate 80 per 100 mg of fusion protein.

### Terms

As used herein, and unless stated otherwise, each of the following terms shall have the definition set forth below.

As used herein, “TV-1106” is an albumin-hGH fusion protein whose amino acid sequence is set forth as SEQ ID NO: 1. TV-1106 is a contiguous protein comprised of human serum albumin (HSA) and human growth hormone (hGH). The HSA moiety confers an extended half-life, while the hGH domain confers the pharmacological properties for the treatment of growth hormone deficiency. TV-1106 is a single polypeptide chain with a molecular mass of approximately 88.5 comprising residues 1-585 corresponding to the mature form of HSA, residues 586-776 corresponding to the mature form of hGH. This sequence is the same as the corresponding native, mature, wild-type sequences of these proteins—no mutations or linkers have been introduced in HSA, hGH or at the junction of between them in TV-1106. TV-1106 can be produced by fermentation and purified by filtration and chromatography processes. (Osborn 2002). TV-1106 substance used to produce a formulation described herein may contain trace amounts of polysorbate 80 introduced during fermentation, e.g., 0.5-2 mg per 100 mg of TV-1106.

As used herein, a “buffer” is a substance which by its presence in solution increases the amount of acid or alkali that must be added to cause unit change in pH. A buffer may include but is not limited to a formulation buffer or a buffer composition.

As used herein, “treating” a disorder, condition, or disease shall mean slowing, stopping, inhibiting or reversing the disorder's progression, and/or ameliorating, lessening, alleviating or removing symptoms of the disorder. Thus, treating a disorder encompasses reversing the disorder's progression, including up to the point of eliminating the disorder itself. “Ameliorating” or “alleviating” a disorder, condition, or disease as used herein shall mean to relieve or lessen the symptoms of that disorder, condition, or disease.

As used herein, “purity,” as in purity of a pharmaceutical composition comprising TV-1106, refers to the relative amount of TV-1106 that is not degraded, is monomeric, and is in its native conformation. Purity may be measured by size exclusion high performance liquid chromatography (SE-HPLC), hydrophobic interaction high performance liquid chromatography (HI-HPLC), sodium dodecylsylfate polyacramide gel electrophoresis (SDS-PAGE), or any other method known in the art, and may be expressed as a percentage.

“Pharmaceutically acceptable excipient” refers to a carrier or excipient that is suitable for use with humans without undue adverse side effects (such as toxicity, irritation, and allergic response) commensurate with a reasonable benefit/risk ratio. It can be a pharmaceutically acceptable solvent, suspending agent or vehicle for delivering the instant compositions to the patient. The carrier may be liquid or solid and is selected with the planned manner of administration in mind. Exemplary excipients suitable for use in the invention include sucrose, trehalose, mannitol, glycerol, surfactants such as polysorbates, buffers, salts such as sodium chloride, alcohols, and the like. Exemplary polysorbates include polysorbate 20, polysorbate 40, polysorbate 60, and polysorbate 80. In some embodiments, the pharmaceutically acceptable excipient comprises phosphate, mannitol, trehalose, and optionally contains polysorbate 80.

By any range disclosed herein, it is meant that all hundredth, tenth and integer unit amounts within the range are specifically disclosed as part of the invention. Thus, for example, 2-200 mg/mL means that 2.01, 2.02 . . . 200.00; 2.1, 2.2 . . . 200.0; and 2, 3 . . . 200 mg/mL unit amounts are included as embodiments of this invention.

List of Abbreviations

### hGH Human Growth Hormone

### HPLC High Performance Liquid Chromatography

### SEC-HPLC Size Exclusion High Performance Liquid Chromatography

### RP-HPLC Reversed-Phase High Performance Liquid Chromatography

### EE-HPLC Ion Exchange High Performance Liquid Chromatography

The specific embodiments and examples described herein are illustrative, and many variations can be introduced on these embodiments and examples without departing from the spirit of the disclosure or from the scope of the appended claims. Elements and/or features of different illustrative embodiments and/or examples may be combined with each other and/or substituted for each other within the scope of this disclosure and appended claims.

For the foregoing embodiments, each embodiment disclosed herein is contemplated as being applicable to each of the other disclosed embodiments.

All combinations and sub-combinations of each of the various elements of the methods and embodiments described herein are envisaged and are within the scope of the invention.

This invention will be better understood by reference to the Examples which follow, which are set forth to aid in an understanding of the subject matter but are not intended to, and should not be construed to, limit in any way the claims which follow thereafter.

### Example 1

### TV-1106 Liquid Formulation Development

### Forced Degradation Studies

Forced degradation studies were performed to determine the effect of different pH conditions on TV-1106.

Increased amounts of isosasparte residues were observed in base-stressed samples as measured by the ISOQUANT® Isoaspartate Detection Kit (Promega). The ISOQUANT® Isoaspartate Detection Kit is an assay which detects isoaspartate residues via protein isoaspartyl methyltransferase (PMT)-catalyzed generation of S-adenosyl homocysteine (SAH). The ISOQUANT® assay detects isoaspartate residues which result from isomerization of aspartate residues and deamidation of asparagine residues. ISOQUANT® assay results expressed in the examples of this application as XY % mean that 0.XY moles of isoaspartate residues were determined to be present per mole of protein assayed. For example, an ISOQUANT® result reported as 12% means that 0.12 moles of isoaspartate residues were observed per mole of protein analyzed. The amount of isoaspartate residues observed suggested deamidation/isomerization at multiple sites.

RP, SEC, and IE-HPLC analyses indicated that TV-1106 stability is affected at high and low pH.

### The Effects of pH and Ionic Strength on TV-1106 Stability

The stability of TV-1106 in SPF buffer (2 mM citric acid, phosphoric acid and Tris base) containing 25, 50, or 100 mM sodium chloride (NaCl) over the range of pH 5.0 to 7.5 in 0.5 unit increments was evaluated.

On the whole, TV-1106 samples prepared near pH 6.5 exhibited the greatest stability (as a measure of sample purity and TV-1106 recovery determined by RP-, SEC-, and IE-HPLC) following seven days incubation at 32° C. and seven days shear stress at ambient laboratory conditions. The data indicated a slightly higher level of recovery and purity levels in the stressed TV-1106 samples (prepared in the pH range of 6.0 to 7.0) with the addition of 25 and 50 mM NaCl.

ANOVA results showed that sample pH had a significant effect on the purity and recovery of TV-1106 when subjected to 32° C. temperature stress or shear stress as determined by RP-, SEC- and EE-HPLC.

### The Effects of pH and Buffer Species on TV-1106 Stability

The stability of TV-1106 as a function of buffer species over the range of pH 6.2 to 7.2 was evaluated as TV-1106 samples prepared near pH 6.5 were observed in the previous set of experiments to exhibit the greatest stability as measured by RP-, SEC-, and IE-HPLC. Polypropylene tubes containing 4 mg/mL TV-1106 buffer and pH test samples were stored at 32° C. and subjected to shear stress (rotation at 12 rpm) at ambient laboratory conditions over the course of ten days.

RP-, SEC-, and IE-HPLC analysis was carried out at t=0 and after four (t=4) and ten days (t=10) incubation at the two stress conditions.

### RP-HPLC Results

TABLE 1
Effect of pH and buffer on TV-1106 stability: RP-HPLC results

| | | TV-1106 conc. (mg/mL), % purity | | | | |
|---|---|---|---|---|---|---|
| Sample Composition | | | 32° C., incubation | | Shaking Stress | |
| (measured pH value) | | t = 0 | t = 4 days | t = 10 days | t = 4 days | t = 10 days |
| Sample Control-CF, pH 7.17 | | 3.7, 92.0 | 3.6, 86.6 | 3.3, 83.0 | 3.7, 88.0 | 3.2, 82.2 |
| 5 mM NaPhosphate | pH 6.58 | 4.1, 93.8 | 3.9, 91.3 | 3.6, 89.0 | 4.0, 90.1 | 3.2, 79.8 |
| | pH 6.67 | 4.2, 93.6 | 3.8, 86.6 | 0.1, 3.7 | 4.2, 91.6 | 3.5, 83.8 |
| | pH 6.82 | 4.1, 93.8 | 3.8, 86.8 | 1.2, 32.3 | 4.1, 91.7 | 3.3, 80.9 |
| | pH 6.91 | 4.1, 94.0 | 4.0, 91.8 | 3.8, 90.7 | 4.1, 92.3 | 3.8, 89.4 |
| | pH 7.02 | 4.2, 93.7 | 3.9, 83.9 | 1.4, 37.1 | 4.1, 91.5 | 3.4, 80.8 |
| 20 mM Glycine | pH 6.63 | 4.3, 93.5 | 3.8, 82.2 | 2.7, 60.9 | 4.1, 91.1 | 3.4, 79.4 |
| | pH 6.70 | 4.5, 93.5 | 4.3, 91.2 | 3.9, 87.4 | 4.2, 89.5 | 3.5, 79.5 |
| | pH 6.76 | 4.3, 93.7 | 3.8, 81.2 | 2.6, 57.6 | 4.3, 90.5 | 3.7, 83.3 |
| | pH 6.76 | 4.4, 93.6 | 3.9, 80.8 | 2.5, 56.5 | 4.3, 91.0 | 3.6, 79.8 |
| | pH 6.73 | 4.4, 93.8 | 3.8, 78.7 | 2.4, 52.3 | 4.0, 85.7 | 3.2. 72.1 |
| 20 mM Histidine | pH 6.51 | 4.0, 92.8 | 4.0, 90.8 | 3.8, 90.0 | 3.9, 89.8 | 3.3, 79.6 |
| | pH 6.68 | 4.2, 92.9 | 4.2, 90.8 | 3.9, 90.7 | 4.1, 90.1 | 3.6, 85.0 |
| | pH 6.87 | 4.1, 92.9 | 4.1, 91.1 | 3.9, 90.9 | 4.0, 90.1 | 3.6, 83.0 |
| | pH 7.03 | 4.2, 92.7 | 4.0, 89.0 | 3.9, 90.3 | 4.0, 88.1 | 3.2, 76.4 |
| | pH 7.19 | 4.2, 92.7 | 4.2, 90.4 | 3.6, 80.8 | 4.1, 91.0 | 3.8, 88.6 |
| 5 mM NaPhosphate + | pH 6.53 | 4.2, 93.5 | 3.2, 66.9 | 1.3, 28.1 | 3.7, 83.4 | 2.1, 52.0 |
| 20 mM Glycine | pH 6.72 | 4.2, 93.3 | 3.1, 66.3 | 1.2, 25.4 | 4.0, 86.8 | 2.4, 55.6 |
| | pH 6.84 | 4.3, 93.5 | 3.2, 67.0 | 1.2, 25.6 | 3.8, 82.7 | 2.3, 51.3 |
| | pH 6.91 | 4.3, 92.9 | 3.3, 67.1 | 1.2, 26.4 | 4.1, 88.0 | 2.7, 60.4 |
| | pH 7.02 | 4.3, 93.4 | 3.5, 70.1 | 1.4, 31.0 | 3.8, 82.8 | 2.3, 53.2 |
| 5 mM NaPhosphate + | pH 6.28 | 4.3, 93.2 | 1.3, 25.3 | 0.1, 0.9 | 3.7, 82.5 | 0.8, 20.9 |
| 20 mM Histidine | pH 6.49 | 4.3. 92.8 | 2.2, 43.4 | 0.1, 2.4 | 3.8, 83.0 | 1.1, 25.4 |
| | pH 6.71 | 4.6, 92.7 | 3.1, 60.8 | 0.5, 12.0 | 4.3, 87.8 | 1.9, 41.8 |
| | pH 6.84 | 4.6, 92.6 | 3.9, 74.8 | 1.4, 30.4 | 4.2, 83.6 | 1.8, 40.8 |
| | pH 6.98 | 4.5, 92.3 | 3.7, 74.7 | 1.4, 31.0 | 4.2, 85.7 | 1.8, 40.3 |

After ten days incubation at 32° C., the highest stability (purity and recovery levels) was observed for the TV-1106 samples formulated with 20 mM histidine and 25 mM sodium chloride over the range of pH 6.5 to 7.0.

TV-1106 sample purity and recovery levels were highest for samples formulated with either 20 histidine or 5 mM sodium phosphate (both containing 25 mM sodium chloride) over the range of pH 6.5 to 7.2 following ten days shear stress at ambient laboratory conditions.

The result of the ANOVA showed that the type of buffer had a significant effect on the purity (F=17.26, p-value <0.0001) and TV-1106 recovery (F=17.25, p-value <0.0001) of TV-1106 when subjected to 32° C. temperature stress. The buffer type also had a significant effect on the purity (F=71.08, p-value <0.0001) and TV-1106 recovery (F=42.45, p-value <0.0001) on TV-1106 test samples subjected to shear stress; moreover, the effect of sample pH was also significant on TV-1106 recovery (F=8.15, p-value 0.0007) when subjected to shear stress.

### RP-HPLC Results

TABLE 2
Effect of pH and buffer on TV-1106 stability: SEC-HPLC results

| | | TV-1106 conc. (mg/mL), % purity | | | | |
|---|---|---|---|---|---|---|
| Sample Composition | | | 32° C. incubation | | Shaking Stress | |
| (measured pH value) | | t = 0 | t = 4 days | t = 10 days | t = 4 days | t = 10 days |
| Sample Control-CF, pH 7.17 | | 3.8, 98.4 | 4.0, 98.3 | 3.8, 98.3 | 3.5, 95.6 | 3.3, 87.4 |
| 5 mM NaPhosphate | pH 6.58 | 4.2, 98.6 | 4.2, 98.7 | 4.1, 98.2 | 4.0, 97.7 | 3.9, 97.2 |
| | pH 6.67 | 4.2, 98.8 | 4.2, 98.5 | 2.4, 65.6 | 4.3, 98.8 | 4.0, 98.3 |
| | pH 6.82 | 4.2, 98.6 | 4.2, 98.3 | 3.5, 89.4 | 4.4, 98.6 | 3.9, 98.1 |
| | pH 6.91 | 4.2, 98.6 | 4.2, 98.7 | 4.1, 98.5 | 4.2, 98.0 | 3.9, 98.0 |
| | pH 7.02 | 4.2, 98.6 | 4.2, 98.2 | 3.7, 91.0 | 4.3, 98.1 | 3.9, 97.4 |
| 20 mM Glycine | pH 6.63 | 4.4, 98.5 | 4.3, 97.2 | 4.2, 95.3 | 4.2, 98.0 | 4.1, 97.3 |
| | pH 6.70 | 4.4, 98.5 | 4.4, 98.5 | 4.3, 97.5 | 4.2, 98.2 | 4.2, 97.5 |
| | pH 6.76 | 4.5, 98.5 | 4.3, 97.3 | 4.2, 95.7 | 4.4, 98.6 | 4.2, 98.0 |
| | pH 6.76 | 4.5, 98.5 | 4.2, 97.3 | 4.3, 95.5 | 4.4, 98.5 | 4.2, 98.0 |
| | pH 6.73 | 4.5, 98.5 | 4.1, 97.1 | 4.3, 95.4 | 4.4, 97.4 | 4.2, 96.9 |
| 20 mM Histidine | pH 6.51 | 4.2, 98.4 | 4.0, 98.3 | 4.2, 98.1 | 4.1, 97.3 | 3.9, 96.3 |
| | pH 6.68 | 4.3, 98.5 | 4.1, 98.6 | 4.3, 98.5 | 4.2, 97.6 | 4.0, 97.3 |
| | pH 6.87 | 4.3, 98.5 | 4.1, 98.6 | 4.2, 98.6 | 4.1, 97.7 | 4.0, 97.5 |
| | pH 7.03 | 4.3, 98.7 | 4.0, 98.8 | 4.2, 98.8 | 0.7, 95.4 | 4.0, 98.3 |
| | pH 7.19 | 4.2, 98.7 | 4.1, 98.8 | 4.2, 97.7 | 4.1, 98.4 | 4.0, 98.3 |
| 5 mM NaPhosphate + | pH 6.53 | 4.3, 98.0 | 4.1, 96.0 | 4.0, 92.6 | 4.1, 96.6 | 3.9, 96.0 |
| 20 mM Glycine | pH 6.72 | 4.3, 98.4 | 4.2, 96.2 | 4.0, 92.8 | 4.2, 97.5 | 4.0, 96.8 |
| | pH 6.84 | 4.4, 98.6 | 4.2, 96.3 | 4.0, 93.0 | 4.2, 96.9 | 4.0, 96.3 |
| | pH 6.91 | 4.4, 98.5 | 4.3, 96.3 | 4.0, 93.2 | 4.1, 98.1 | 4.1, 97.3 |
| | pH 7.02 | 4.4, 98.6 | 4.2, 96.4 | 4.0, 93.4 | 4.0, 97.0 | 4.0, 96.3 |
| 5 mM NaPhosphate + | pH 6.28 | 4.5, 98.4 | 4.3, 96.0 | 3.7, 87.3 | 4.3, 97.7 | 3.9, 92.3 |
| 20 mM Histidine | pH 6.49 | 4.5, 98.5 | 4.3, 96.5 | 4.0, 92.4 | 4.5, 97.8 | 4.0, 94.5 |
| | pH 6.71 | 4.6, 98.6 | 4.4, 97.0 | 4.1, 92.6 | 4.6, 98.4 | 4.2, 96.1 |
| | pH 6.84 | 4.7, 98.5 | 4.5, 97.4 | 4.3, 94.2 | 4.7, 98.2 | 4.3, 95.8 |
| | pH 6.98 | 4.6, 98.6 | 4.4, 97.4 | 4.2, 94.0 | 4.6, 97.7 | 4.2, 95.6 |

After ten days incubation at 32° C., the highest stability (purity and recovery levels) was observed for the TV-1106 samples formulated with 20 mM histidine and 25 mM sodium chloride over the range of pH 6.5 to 7.2.

TV-1106 sample purity and recovery levels were highest for samples formulated with 20 mM histidine, 20 mM glycine, or 5 mM sodium phosphate (each containing 25 mM sodium chloride) over the range of pH 6.6 to 7.2 following ten days shear stress at ambient laboratory conditions. The result of the ANOVA showed that the type of buffer had a significant effect on the purity (F=5.58, p-value 0.0038) and TV-1106 recovery (F=7.89, p-value 0.0006) of TV-1106 when subjected to 32° C. temperature stress. Analysis of the shear stress data indicated that buffer type and pH had a significant effect on the purity (F=11.54, p-value 0.0003 and F=6.39, p-value 0.0021, respectively) and TV-1106 recovery (F=11.63, p-value 0.0003 and F=6.66, p-value 0.0017, respectively).

### IE-HPLC Results

TABLE 3
Effect of pH and buffer on TV-1106 stability: IE-HPLC results

| | | TV-1106 conc. (mg/mL), % purity | | | | |
|---|---|---|---|---|---|---|
| Sample Composition | | | 32° C., incubation | | Shaking Stress | |
| (measured pH value) | | t = 0 | t = 4 days | t = 10 days | t = 4 days | t = 10 days |
| Sample Control-CF, pH 7.17 | | 3.9, 82.4 | 2.5, 74.6 | 2.7, 61.3 | 3.3, 79.0 | 2.8, 74.9 |
| 5 mM NaPhosphate | pH 6.58 | 3.9, 82.2 | 4.0, 81.5 | 3.6, 75.4 | 3.7, 80.4 | 3.5, 78.1 |
| | pH 6.67 | 3.9, 82.5 | 3.1, 73.6 | 0.1, 2.2 | 3.9, 80.3 | 3.7, 77.7 |
| | pH 6.82 | 4.0, 83.0 | 3.6, 73.9 | 1.4, 31.4 | 3.9, 80.6 | 3.5, 74.7 |
| | pH 6.91 | 4.0, 83.6 | 3.1, 76.0 | 3.5, 72.9 | 3.8, 80.8 | 3.7, 78.7 |
| | pH 7.02 | 3.9, 82.6 | 2.8, 74.8 | 1.6, 35.6 | 3.9, 81.1 | 3.4, 73.2 |
| 20 mM Glycine | pH 6.63 | 4.0, 82.6 | 3.4, 76.6 | 2.8, 57.3 | 4.0, 82.2 | 3.5, 72.1 |
| | pH 6.70 | 4.0, 83.1 | 3.4, 77.7 | 3.2, 61.9 | 4.1, 82.7 | 3.5, 71.3 |
| | pH 6.76 | 4.0, 83.4 | 3.6, 76.0 | 3.0, 57.3 | 4.1, 82.1 | 3.3, 67.5 |
| | pH 6.76 | 4.0, 83.2 | 3.8, 76.1 | 2.9, 55.9 | 4.1, 82.0 | 3.3, 66.0 |
| | pH 6.73 | 4.0, 83.1 | 3.5, 75.7 | 2.9, 54.4 | 4.0, 82.7 | 3.3, 67.4 |
| 20 mM Histidine | pH 6.51 | 4.0, 84.7 | 3.4, 81.6 | 3.6, 70.8 | 4.0, 77.4 | 3.4, 74.1 |
| | pH 6.68 | 4.0, 84.4 | 3.4, 81.4 | 3.8, 72.3 | 4.0, 78.1 | 3.7, 77.0 |
| | pH 6.87 | 3.9, 85.4 | 3.7, 80.2 | 3.5, 68.8 | 4.0, 78.4 | 3.6, 74.9 |
| | pH 7.03 | 4.0, 85.6 | 3.7, 78.3 | 3.3, 65.5 | 3.9, 78.7 | 3.4, 72.3 |
| | pH 7.19 | 4.0, 85.3 | 3.5, 77.5 | 3.2, 62.3 | 3.9, 80.8 | 3.5, 73.8 |
| 5 mM NaPhosphate + | pH 6.53 | 4.0, 85.4 | 3.1, 73.4 | 2.4, 46.0 | 3.9, 80.5 | 3.0, 65.2 |
| 20 mM Glycine | pH 6.72 | 4.1, 85.2 | 3.2, 73.7 | 2.4, 45.6 | 4.1, 81.6 | 3.2, 68.4 |
| | pH 6.84 | 3.9, 85.2 | 3.0, 72.5 | 2.2, 42.2 | 4.0, 79.4 | 3.1, 64.6 |
| | pH 6.91 | 4.0, 85.4 | 2.7, 73.7 | 2.1, 41.4 | 4.0, 80.8 | 3.3, 67.8 |
| | pH 7.02 | 4.0, 85.9 | 2.5, 73.6 | 2.3, 45.5 | 3.7, 79.2 | 3.2, 65.4 |
| 5 mM NaPhosphate + | pH 6.28 | 3.9, 86.7 | 3.0, 62.2 | 1.3, 26.5 | 3.2, 80.6 | 2.8, 59.2 |
| 20 mM Histidine | pH 6.49 | 4.0, 86.4 | 2.9, 71.0 | 1.9, 38.3 | 3.6, 81.6 | 2.8, 58.7 |
| | pH 6.71 | 3.9, 86.7 | 3.3, 73.3 | 2.4, 48.0 | 3.6, 82.8 | 3.2, 62.5 |
| | pH 6.84 | 4.3, 87.0 | 3.8, 75.2 | 2.5, 49.6 | 4.0, 81.0 | 3.2, 64.8 |
| | pH 6.98 | 4.3, 86.0 | 3.4, 74.7 | 2.4, 49.4 | 3.8, 82.0 | 3.0, 62.3 |

After ten days incubation at 32° C., the highest stability (purity and recovery levels) was observed for the TV-1106 samples formulated with 20 mM histidine and 25 mM sodium chloride over the range of pH 6.5 to 7.0.

TV-1106 sample purity and recovery levels were highest for samples formulated with either 20 histidine or 5 mM sodium phosphate (both containing 25 mM sodium chloride) over the range of pH 6.7 to 7.2 following ten days shear stress at ambient laboratory conditions.

The result of the ANOVA showed that the type of buffer had a significant effect on the purity (F=4.68, p-value 0.0085) and TV-1106 recovery (F=8.40, p-value 0.0004) of TV-1106 when subjected to 32° C. temperature stress. The buffer type also had a significant effect on the purity (F=37.05, p-value <0.0001) and TV-1106 recovery (F=12.77, p-value 0.0002) on TV-1106 test samples subjected to shear stress; moreover, the effect of sample pH was also significant on TV-1106 recovery (F=3.28, p-value 0.0307) when subjected to shear stress.

### Conclusion

On the whole, TV-1106 samples prepared in 20 mM histidine and 25 mM sodium chloride over the pH range 6.5 to 7.2 exhibited the greatest stability (as a measure of sample purity and TV-1106 determined by RP-, SEC-, and RP-HPLC) following ten days incubation at 32° C. TV-1106 samples subjected to shear stress at ambient laboratory conditions demonstrated the highest stability in preparations containing 20 mM histidine (and 25 mM sodium chloride) or 5 mM sodium phosphate (and 25 mM sodium chloride) over the range of pH 6.5 to 7.2.

### The Effects of pH, Buffer, and Tonicity Adjuster on TV-1106 Stability

A study was conducted to evaluate the effects of pH, buffer type, and tonicity adjuster (e.g., salt, carbohydrate, or polyalcohol) on TV-1106 stability the range of pH 6.2 to 7.2.

No clear benefit could be observed between the various tonicity adjusters tested following one week incubation at 32° C. An optimal tonicity adjuster (e.g., salt, carbohydrate, or polyalcohol) was not uncovered.

### Excipient Combination Screening 1

A study was conducted to evaluate the effects of combinations of excipients on TV-1106 stability at pH 6.5.

A 3 mL volume of TV-1106 bulk drug product was dialyzed against 100-fold appropriate buffer without additional surfactant at 5° C. Sample buffer was changed 3× over the course of 24 hours. Following dialysis, aliquots of the dialyzed buffer samples were removed and the requisite amount of TWEEN®-80 was added to a final concentration of 0.03% v/v. A 2 mL volume of TV-1106 stocks were concentrated to roughly 1 mL in 10K MWCO Amicon spin filters to a nominal concentration of 100 mg/mL.

Aliquots of the buffered solutions were placed in screw top polypropylene tubes and stored at 5° C., 25° C. and 32° C. for up to 14 days. RP-, SEC-, and IE-HPLC analysis was carried out immediately following sample preparation and after three (t=3), seven (t=7), and fourteen (t=14) days incubation at the appropriate stress condition.

### RP-HPLC Results

TABLE 4
Excipient combination screening on TV-1106: RP-HPLC results

| | | TV-1106 conc. (mg/mL), % purity | | | | | | |
|---|---|---|---|---|---|---|---|---|
| | | | 5° C. incubation | | 25° C. incubation | | 32° C. incubation | |
| Sample Composition | | t = 0 | t = 7 days | t = 14 days | t = 3 days | t = 7 days | t = 3 days | t = 7 days |
| Sample Control-CF, pH 7.17 | | 62.5, 92.1 | 62.9, 91.0 | 61.4, 90.5 | 62.0, 90.4 | 57.3, 88.4 | 60.2, 88.9 | 64.1, 86.1 |
| 10 mM Histidine, | 100 mM NaCl | 62.1, 92.0 | 59.8, 91.1 | 59.8, 91.0 | 56.7, 90.9 | 57.5, 89.7 | 60.1, 89.9 | 58.8, 88.5 |
| pH 6.5 | 100 mM NaCl + | 68.2, 91.8 | 69.1, 91.0 | 67.7, 90.8 | 69.0, 90.1 | 71.1, 89.6 | 73.1, 89.6 | 64.5, 88.2 |
| | 125 mM Trehalose | | | | | | | |
| | 100 mM NaCl + | 60.3, 91.9 | 61.8, 91.1 | 63.5, 91.0 | 61.3, 91.1 | 61.4, 89.8 | 66.9, 89.8 | 62.7, 87.9 |
| | 125 mM Mannitol | | | | | | | |
| | 125 mM Trehalose + | 57.0, 91.8 | 55.8, 91.0 | 57.0, 90.7 | 55.3, 90.6 | 52.7, 89.0 | 56.8, 88.2 | 54.6, 86.2 |
| | 125 mM Mannitol | | | | | | | |
| 10 mM Histidine + | 100 mM NaCl | 56.4, 92.0 | 55.2, 91.0 | 59.6, 90.8 | 57.3, 90.8 | 58.8, 89.5 | 57.0, 89.7 | 59.7, 88.4 |
| 0.03% Tween-80, | 100 mM NaCl + | 66.6, 91.9 | 70.7, 91.0 | 68.9, 90.8 | 65.0, 90.7 | 67.6, 89.4 | 70.7, 89.3 | 69.2, 87.8 |
| pH 6.5 | 125 mM Trehalose | | | | | | | |
| | 100 mM NaCl + | 62.7, 91.3 | 63.2, 91.0 | 62.4, 90.8 | 59.6, 90.9 | 61.6, 89.6 | 63.6, 89.4 | 62.2, 87.0 |
| | 125 mM Mannitol | | | | | | | |
| | 125 mM Trehalose + | 52.0, 91.6 | 52.6, 91.0 | 55.7, 90.7 | 53.5, 90.3 | 54.0, 88.7 | 53.7, 88.2 | 52.2, 86.2 |
| | 125 mM Mannitol | | | | | | | |

| | | | | | | | |
|---|---|---|---|---|---|---|---|
| 10 mM His, pH 6.5 + 250 mM Trehalose | 100.9, 91.7 | 101.4, 91.3 | 102.9, 91.2 | 87.6, 91.4 | 94.1, 89.8 | 91.3, 89.5 | 98.4, 87.2 |
| 10 mM His, pH 6.5 + 150 mM NaCl | 101.0, 91.5 | 108.5, 91.5 | 110.5, 91.4 | 78.2, 91.7 | 99.0, 89.8 | 97.7, 90.5 | 105.0, 88.9 |
| Stock 4098A (Phos/Man/Tre, pH 7.2) | 60.2, 92.0 | 65.0, 91.4 | 65.8, 90.9 | 63.8, 90.9 | 58.8, 89.1 | 63.9, 89.3 | 59.8, 86.9 |
| Stock 4098B (His/250 mM Trehalose, | 59.3, 92.0 | 67.5, 91.3 | 65.1, 90.9 | 61.0, 91.1 | 56.3, 90.0 | 70.2, 89.0 | 59.9, 88.0 |
| pH 6.5) | | | | | | | |
| Stock 4098C (His/150 mM NaCl, pH 6.5) | 59.1, 92.2 | 66.9, 91.3 | 65.5, 91.6 | 60.6, 91.4 | 60.1, 90.0 | 65.4, 90.0 | 64.6, 88.4 |

TV-1106 samples containing 100 mM NaCl were the most stable when compared to the trehalose+mannitol buffer samples following incubation at 25° C. and 32° C. for seven days. Moreover, samples which did not contain 0.03% TWEEN®-80 were significantly more pure than those samples that did upon incubation at 25° C. for seven days.

### SEC-HPLC Results

TABLE 5
Excipient combination screening on TV-1106: SEC-HPLC results

| | | TV-1106 conc. (mg/mL), % purity | | | | | | |
|---|---|---|---|---|---|---|---|---|
| | | | 5° C. incubation | | 25° C. incubation | | 32° C. incubation | |
| Sample Composition | | t = 0 | t = 7 days | t = 14 days | t = 3 days | t = 7 days | t = 3 days | t = 7 days |
| Sample Control-CF, pH 7.17 | | 60.4, 99.4 | 57.4, 99.3 | 61.2, 96.9 | 61.8, 99.1 | 59.2, 99.0 | 63.8, 99.0 | 58.9, 98.8 |
| 10 mM Histidine, | 100 mM NaCl | 54.1, 99.5 | 53.9, 99.3 | 58.3, 97.0 | 58.2, 99.4 | 53.9, 99.1 | 62.2, 99.1 | 53.9, 99.0 |
| pH 6.5 | 100 mM NaCl + | 66.5, 99.5 | 64.6, 99.4 | 70.6, 97.1 | 69.4, 99.4 | 65.6, 99.0 | 74.2, 99.2 | 68.7, 98.9 |
| | 125 mM Trehalose | | | | | | | |
| | 100 mM NaCl + | 59.5, 99.5 | 58.6, 99.4 | 60.8, 97.0 | 67.1, 99.3 | 58.1, 99.1 | 66.5, 99.2 | 60.6, 99.0 |
| | 125 mM Mannitol | | | | | | | |
| | 125 mM Trehalose + | 54.5, 99.1 | 52.1, 98.5 | 55.3, 95.2 | 54.7, 97.7 | 51.4, 97.1 | 54.8, 95.6 | 51.0, 94.4 |
| | 125 mM Mannitol | | | | | | | |
| 10 mM Histidine + | 100 mM NaCl | 56.1, 99.4 | 56.8, 99.2 | 57.1, 95.2 | 60.0, 99.1 | 57.3, 98.8 | 59.4, 99.0 | 58.9, 98.6 |
| 0.03% Tween-80, | 100 mM NaCl + | 66.2, 99.4 | 64.7, 99.1 | 67.2, 96.0 | 71.6, 99.2 | 65.0, 98.9 | 71.3, 99.0 | 70.6, 98.6 |
| pH 6.5 | 125 mM Trehalose | | | | | | | |
| | 100 mM NaCl + | 59.2, 99.4 | 59.9, 99.1 | 61.6, 95.8 | 63.9, 99.3 | 59.9, 98.9 | 63.2, 99.1 | 62.8, 98.7 |
| | 125 mM Mannitol | | | | | | | |
| | 125 mM Trehalose + | 53.4, 98.9 | 53.9, 97.9 | 54.2, 93.9 | 56.6, 97.6 | 52.9, 96.9 | 53.8, 95.8 | 54.0, 94.6 |
| | 125 mM Mannitol | | | | | | | |

| | | | | | | | |
|---|---|---|---|---|---|---|---|
| 10 mM His, pH 6.5 + 250 mM Trehalose | 92.9, 98.7 | 89.7, 98.0 | 92.7, 95.4 | 92.7, 96.9 | 87.6, 96.1 | 86.2, 93.8 | 86.3, 92.1 |
| 10 mM His, pH 6.5 + 150 mM NaCl | 99.3, 99.5 | 98.7, 99.3 | 101.7, 97.8 | 103.7, 99.4 | 101.1, 99.0 | 104.8, 99.2 | 96.6, 98.8 |
| Stock 4098A (Phos/Man/Tre, pH 7.2) | 60.2, 99.4 | 58.4, 99.1 | 63.3, 97.6 | 64.4, 99.2 | 58.9, 99.1 | 62.4, 99.0 | 56.6, 98.1 |
| Stock 4098B (His/250 mM Trehalose, | 59.3, 98.7 | 58.9, 98.3 | 58.0, 95.4 | 63.0, 97.5 | 55.8, 97.0 | 60.2, 95.6 | 57.1, 94.7 |
| pH 6.5) | | | | | | | |
| Stock 4098C (His/150 mM NaCl, pH 6.5) | 59.1, 99.6 | 60.3, 99.5 | 65.9, 97.4 | 68.3, 99.5 | 60.0, 99.3 | 64.1, 99.3 | 60.6, 99.1 |

TV-1106 samples containing 100 mM NaCl were the most stable when compared to the trehalose+mannitol buffer samples following incubation at 5° C., 25° C. and 32° C. for seven days. Moreover, samples which did not contain 0.03% TWEEN®-80 were significantly more stable than those samples that did upon incubation at 5° C. and 25° C. for seven days.

### IE-HPLC Results

TABLE 6
Excipient combination screening on TV-1106: IE-HPLC result

| | | TV-1106 conc. (mg/mL), % purity | | | | | | |
|---|---|---|---|---|---|---|---|---|
| | | | 5° C. incubation | | 25° C. incubation | | 32° C. incubation | |
| Sample Composition | | t = 0 | t = 7 days | t = 14 days | t = 3 days | t = 7 days | t = 3 days | t = 7 days |
| Sample Control-CF, pH 7.17 | | 62.6, 93.0 | 64.2, 92.5 | 64.6, 92.8 | 63.9, 91.6 | 62.0, 88.7 | 61.9, 88.5 | 56.0, 81.9 |
| 10 mM Histidine, | 100 mM NaCl | 59.1, 93.2 | 56.9, 92.6 | 60.2, 93.2 | 58.9, 92.5 | 57.7, 90.4 | 62.5, 90.9 | 54.4, 86.5 |
| pH 6.5 | 100 mM NaCl + | 68.4, 93.0 | 66.4, 92.6 | 73.5, 92.7 | 72.6, 92.7 | 69.2, 90.5 | 73.8, 90.7 | 67.0, 86.9 |
| | 125 mM Trehalose | | | | | | | |
| | 100 mM NaCl + | 60.5, 92.9 | 60.7, 92.7 | 58.3, 92.8 | 64.9, 92.7 | 61.5, 90.3 | 67.4, 90.9 | 59.9, 86.1 |
| | 125 mM Mannitol | | | | | | | |
| | 125 mM Trehalose + | 55.4, 93.0 | 53.7, 93.8 | 57.3, 93.3 | 58.2, 93.0 | 54.6, 92.0 | 58.0, 91.5 | 52.5, 89.5 |
| | 125 mM Mannitol | | | | | | | |
| 10 mM Histidine + | 100 mM NaCl | 56.4, 93.2 | 56.6, 92.7 | 60.7, 92.5 | 60.7, 92.9 | 60.4, 89.9 | 59.8, 90.7 | 56.4, 87.4 |
| 0.03% Tween-80, | 100 mM NaCl + | 66.8, 92.9 | 69.0, 92.7 | 71.3, 92.6 | 72.2, 92.6 | 69.1, 90.5 | 73.0, 91.0 | 68.6, 87.7 |
| pH 6.5 | 125 mM Trehalose | | | | | | | |
| | 100 mM NaCl + | 58.9, 93.0 | 61.2, 92.8 | 64.0, 92.4 | 64.4, 91.9 | 62.3, 90.3 | 66.4, 90.9 | 61.3, 86.8 |
| | 125 mM Mannitol | | | | | | | |
| | 125 mM Trehalose + | 54.1, 93.4 | 55.5, 93.5 | 57.0, 93.0 | 56.8, 92.6 | 58.2, 92.4 | 58.5, 91.4 | 52.3, 89.8 |
| | 125 mM Mannitol | | | | | | | |

| | | | | | | | |
|---|---|---|---|---|---|---|---|
| 10 mM His, pH 6.5 + 250 mM Trehalose | 101.6, 93.2 | 94.8, 94.2 | 98.5, 93.2 | 97.1, 92.6 | 91.3, 92.4 | 96.5, 91.9 | 86.2, 90.2 |
| 10 mM His, pH 6.5 + 150 mM NaCl | 109.0, 93.5 | 101.1, 93.2 | 106.8, 92.7 | 111.3, 92.6 | 104.3, 89.9 | 107.0, 90.9 | 100.8, 86.6 |
| Stock 4098A (Phos/Man/Tre, pH 7.2) | 59.0, 92.8 | 63.1, 93.5 | 65.2, 92.7 | 67.3, 91.8 | 58.7, 88.0 | 64.2, 88.9 | 55.0, 82.3 |
| Stock 4098B (His/250 mM Trehalose, | 57.0, 93.0 | 61.2, 93.0 | 67.8, 93.8 | 68.2, 93.0 | 57.8, 92.7 | 64.7, 91.9 | 59.1, 90.4 |
| pH 6.5) | | | | | | | |
| Stock 4098C (His/150 mM NaCl, pH 6.5) | 56.9, 93.5 | 62.9, 92.8 | 65.5, 93.0 | 71.2, 92.3 | 60.4, 90.9 | 68.4, 90.5 | 58.1, 86.0 |

TV-1106 samples containing both trehalose+mannitol were the most stable when compared to the other formulations following incubation at 5° C., 25° C. and 32° C. for seven days. Moreover, samples which did contain 0.03% TWEEN®-80 were significantly more pure than those samples that did not upon incubation at 32° C. for seven days.

### Conclusion

A marked preservation of TV-1106 purity was observed in test samples containing 100 mM NaCl as determined by RP- and SEC-HPLC analyses. TV-1106 which did not contain 0.03% TWEEN®-80, in comparison to those samples that did contain TWEEN®-80, where found to have higher purity values following incubation at 5° C. and 25° C. The converse was found to be true for test samples incubated at 32° C. Interestingly, EE-HPLC data suggested that TV-1106 samples containing both trehalose+mannitol (no additional NaCl) were the most stable when compared to the other formulations following incubation at 5° C., 25° C. and 32° C. for seven days.

ANOVA results showed that sample buffer containing 100 mM NaCl had a significant effect on the purity as determined by RP- and SEC-HPLC data, whereas the presence of trehalose+mannitol (no additional salt) was found to be the most significant factor contributing to test sample purity as determined by IE-HPLC. The necessity for the addition of TWEEN®-80 to TV-1106 appears to be a temperature-dependent phenomenon in which at lower storage temperatures the need for TWEEN®-80 is unwarranted. No clear indication for the necessity of additional surfactant was discovered.

### Excipient Combination Screening 2

A study was conducted to evaluate the effects of combinations of excipients on TV-1106 stability at pH 6.5. Subsequent to sample preparation, test samples were stressed by incubation at 5° C., 25° C. and 32° C. and were evaluated for purity and recovery via RP, SEC, and EE-HPLC.

Samples were stored at 5° C., 25° C. and 32° C. for 70 days. RP-, SEC-, and EE-HPLC analysis was carried out immediately following sample preparation and after incubation at the appropriate stress condition.

### RP-HPLC Results

TABLE 7
Excipient combination screening #2 on TV-1106: RP-HPLC results

| | | TV-1106 conc. (mg/mL), % purity | | | | | | | | | | |
|---|---|---|---|---|---|---|---|---|---|---|---|---|
| | | | 5° C. incubation | | | | 25° C. incubation | | | 32° C. incubation | | |
| | | | t = 7 | t = 14 | t = 27 | t = 70 | t = 3 | t = 7 | t = 27 | t = 3 | t = 7 | t = 27 |
| Sample Composition | | t = 0 | days | days | days | days | days | days | days | days | days | days |
| Sample Control-CF, pH 7.17 | | 49.0, | 53.7, | 52.8, | 46.9, | 49.7, | 52.5, | 53.6, | 64.4, | 52.5, | 51.6, | 57.0, |
| | | 90.1 | 90.7 | 90.2 | 89.4 | 88.6 | 88.4 | 87.3 | 84.8 | 88.1 | 86.0 | 78.3 |
| 10 mM Histidine, | 50 mM NaCl/50 mM | 66.3, | 67.6, | 71.6, | 68.1, | 67.4, | 69.0, | 72.7, | 81.5, | 67.3, | 70.1, | —* |
| pH 6.5 (0.07% | Mann/50 mM Tre | 91.1 | 90.9 | 90.8 | 90.2 | 89.1 | 89.6 | 88.5 | 83.4 | 88.7 | 87.0 | — |
| Tween-80) | (299 mOsm/kg) | | | | | | | | | | | |
| | 100 mM NaCl/50 mM | 54.6, | 58.2, | 58.4, | 54.3, | 55.3, | 57.2, | 57.5, | 49.4, | 58.6, | 58.2, | 67.6, |
| | Mann/50 mM Tre | 91.3 | 91.2 | 91.1 | 90.4 | 89.5 | 89.8 | 88.8 | 84.8 | 89.1 | 87.2 | 81.0 |
| | (421 mOsm/kg) | | | | | | | | | | | |
| | 50 mM NaCl/125 mM | 64.7, | 67.6, | 71.6, | 65.8, | 65.7, | 68.3, | 70.6, | 70.8, | 68.0, | 70.1, | 82.7, |
| | Mann/50 mM Tre | 91.9 | 90.8 | 90.9 | 90.0 | 89.4 | 89.6 | 88.5 | 83.6 | 89.4 | 87.5 | 78.1 |
| | (429 mOsm/kg) | | | | | | | | | | | |
| | 100 mM NaCl/125 mM | 54.0, | 58.5, | 58.6, | 54.6, | 56.2, | 57.0, | 60.0, | 67.7, | 57.2, | 58.6, | 69.8, |
| | Mann/50 mM Tre | 91.1 | 91.0 | 90.9 | 90.4 | 89.3 | 89.7 | 88.7 | 84.3 | 89.0 | 87.2 | 78.7 |
| | (536 mOsm/kg) | | | | | | | | | | | |
| | 50 mM NaCl/50 mM | 52.0, | 55.0, | 58.2, | 54.4, | 53.0, | 53.8, | 57.1, | 48.6, | 54.7, | 55.8, | 66.8, |
| | Mann/125 mM Tre | 91.1 | 91.0 | 91.0 | 90.5 | 91.0 | 89.9 | 89.1 | 89.1 | 89.2 | 87.4 | 81.7 |
| | (380 mOsm/kg) | | | | | | | | | | | |
| | 100 mM NaCl/50 mM | 51.1, | 55.4, | 57.2, | 52.0, | 52.6, | 53.4, | 58.4, | 56.6, | 54.9, | 55.5, | 72.0, |
| | Mann/125 mM Tre | 91.2 | 91.0 | 90.8 | 90.5 | 89.9 | 89.8 | 89.0 | 84.7 | 89.1 | 87.0 | 79.5 |
| | (541 mOsm/kg) | | | | | | | | | | | |
| | 50 mM NaCl/125 mM | 51.7, | 54.8, | 55.4, | 51.8, | 51.1, | 53.3, | 56.6, | 59.3, | 54.7, | 55.6, | 69.1, |
| | Mann/125 mM Tre | 91.1 | 90.9 | 91.2 | 90.5 | 89.8 | 89.8 | 89.2 | 84.9 | 89.0 | 87.3 | 78.2 |
| | (568 mOsm/kg) | | | | | | | | | | | |
| | 100 mM NaCl/125 mM | 52.4, | 54.5, | 57.6, | 52.4, | 52.9, | 55.3, | 56.9, | 55.5, | 53.2, | 54.7, | 75.7, |
| | Mann/125 mM Tre | 91.0 | 90.6 | 90.9 | 90.5 | 89.8 | 89.6 | 88.9 | 85.1 | 89.1 | 87.4 | 79.5 |
| | (693 mOsm/kg) | | | | | | | | | | | |

The data suggest that following storage at 25° C. high concentrations of trehalose (125 mM) provide significantly more stable conditions (greater purity) than low trehalose concentrations. Following storage at 32° C., low concentrations of mannitol (50 mM) provide significantly more stable conditions (greater purity) than higher mannitol concentrations.

### SEC-HPLC Results

TABLE 8
Excipient combination screening #2 on TV-1106: SEC-HPLC results

| | | TV-1106 conc. (mg/mL), % purity | | | | | | | | | | |
|---|---|---|---|---|---|---|---|---|---|---|---|---|
| | | | 5° C. incubation | | | | 25° C. incubation | | | 32° C. incubation | | |
| | | | t = 6 | t = 14 | t = 25# | t = 70# | t = 3 | t = 6 | t = 25# | t = 3 | t = 6 | t = 26# |
| Sample Composition | | t = 0 | days | days | days | days | days | days | days | days | days | days |
| Sample Control-CF, pH 7.17 | | 50.9, | 45.9, | 48.9, | 51.3, | 52.3, | 51.6, | 48.9, | 73.1, | 51.3, | 46.8, | 68.3, |
| | | 94.9 | 92.0 | 93.9 | 98.4 | 98.1 | 98.6 | 88.4 | 98.0 | 95.9 | 89.0 | 97.5 |
| 10 mM Histidine, | 50 mM NaCl/50 mM | 65.6, | 65.6, | 61.4, | 70.6, | 63.5, | 69.6, | 67.1, | 70.4, | 63.2, | 66.1, | 72.4, |
| pH 6.5 (0.07% | Mann/50 mM Tre | 96.4 | 96.0 | 92.8 | 99.1 | 98.5 | 99.0 | 89.7 | 98.4 | 95.1 | 93.1 | 97.8 |
| Tween-80) | (299 mOsm/kg) | | | | | | | | | | | |
| | 100 mM NaCl/50 mM | 54.0, | 57.2, | 50.0, | 57.1, | 56.4, | 53.9, | 53.7, | 49.1, | 54.9, | 51.7, | 69.3, |
| | Mann/50 mM Tre | 95.0 | 94.7 | 95.6 | 99.0 | 98.6 | 99.1 | 89.6 | 98.8 | 96.0 | 91.5 | 98.6 |
| | (421 mOsm/kg) | | | | | | | | | | | |
| | 50 mM NaCl/125 mM | 67.1, | 65.9, | 70.4, | 68.5, | 69.9, | 65.4, | 64.2, | 72.2, | 55.8, | 65.4, | 53.0*, |
| | Mann/50 mM Tre | 95.7 | 94.9 | 95.4 | 98.8 | 98.2 | 98.6 | 89.8 | 98.2 | 96.2 | 90.3 | 73.8 |
| | (429 mOsm/kg) | | | | | | | | | | | |
| | 100 mM NaCl/125 mM | 53.6, | 53.4, | 58.1, | 55.1, | 56.3, | 55.7, | 52.4, | 67.3, | 56.6, | 53.2, | 77.4, |
| | Mann/50 mM Tre | 95.3, | 95.1 | 95.1 | 99.0 | 98.5 | 98.8 | 89.0 | 98.8 | 96.8 | 92.8 | 98.6 |
| | (536 mOsm/kg) | | | | | | | | | | | |
| | 50 mM NaCl/50 mM | 51.7, | 51.3, | 53.8, | 56.0, | 52.5, | 52.8, | 47.5, | 48.5, | 52.2, | 49.7, | 70.7, |
| | Mann/125 mM Tre | 93.8 | 94.3 | 93.8 | 98.8 | 98.2 | 98.6 | 89.5 | 98.7 | 96.1 | 91.5 | 98.3 |
| | (380 mOsm/kg) | | | | | | | | | | | |
| | 100 mM NaCl/50 mM | 50.1, | 51.5, | 53.8, | 54.6, | 53.3, | 53.3, | 48.8, | 57.6, | 50.7, | 51.2, | 72.0, |
| | Mann/125 mM Tre | 92.4 | 93.6 | 94.3 | 98.9 | 98.4 | 98.7 | 89.4 | 98.8 | 95.5 | 92.3 | 98.4 |
| | (541 mOsm/kg) | | | | | | | | | | | |
| | 50 mM NaCl/125 mM | 49.2, | 49.4, | 52.5, | 55.5, | 53.3, | 51.6, | 49.0, | 60.9, | 51.3, | 50.8, | 72.0, |
| | Mann/125 mM Tre | 90.0 | 93.3 | 93.9 | 98.9 | 98.3 | 98.8 | 89.6 | 98.7 | 96.5 | 93.6 | 98.4 |
| | (568 mOsm/kg) | | | | | | | | | | | |
| | 100 mM NaCl/125 mM | 50.1, | 51.7, | 51.6, | 54.7, | 54.7, | 52.4, | 49.9, | 58.2, | 50.7, | 50.8, | 76.9, |
| | Mann/125 mM Tre | 93.2 | 93.9 | 93.5 | 98.8 | 98.4 | 98.8 | 88.7 | 98.7 | 96.4 | 93.5 | 98.5 |
| | (693 mOsm/kg) | | | | | | | | | | | |

*denotes yellowing of sample;
#denotes samples prepared for analysis by gentle pipetting.
Previous samples were prepared by light vortexing 3-5 seconds.

ANOVA results showed no significant effects due to the various excipient combinations upon storage after 70 days at 5° C. by SEC-HPLC. Following storage at 25° C., high concentrations of sodium chloride (100 mM) provided significantly more stable conditions (greater purity) than lower sodium chloride concentrations

### IE-HPLC Results

TABLE 9
Excipient combination screening #2 on TV-1106: IE-HPLC results

| | | TV-1106 conc. (mg/mL), % purity | | | | | | | | | |
|---|---|---|---|---|---|---|---|---|---|---|---|
| | | | 5° C. incubation | | | | 25° C. incubation | | 32° C. incubation | | |
| | | | t = 7 | t = 14 | t = 28 | t = 73 | t = 3 | t = 7 | t = 3 | t = 7 | t = 28 |
| Sample Composition | | t = 0 | days | days | days | days | days | days | days | days | days |
| Sample Control-CF, pH 7.17 | | 49.2, | 48.5, | 49.4, | 49.4, | 48.6, | 44.5, | 43.8, | 46.2, | 40.0, | 35.3, |
| | | 88.8 | 88.8 | 89.5 | 87.7 | 85.9 | 86.5 | 82.0 | 83.1 | 76.3 | 50.9 |
| 10 mM Histidine, | 50 mM NaCl/50 mM | 65.8, | 70.5, | 68.8, | 72.3, | 70.9, | 69.8, | 67.4, | 67.1, | 64.0, | —* |
| pH 6.5 (0.07% | Mann/50 mM Tre | 93.2 | 93.7 | 93.7 | 92.8 | 91.9 | 93.2 | 90.2 | 91.3 | 86.8 | — |
| Tween-80) | (299 mOsm/kg) | | | | | | | | | | |
| | 100 mM NaCl/50 mM | 54.2, | 55.5, | 57.0, | 58.4, | 54.0, | 55.1, | 55.7, | 56.3, | 52.1, | 58.1, |
| | Mann/50 mM Tre | 93.4 | 94.2 | 94.2 | 93.1 | 92.5 | 93.2 | 89.8 | 91.2 | 85.9 | 68.5 |
| | (421 mOsm/kg) | | | | | | | | | | |
| | 50 mM NaCl/125 mM | 66.7, | 67.5, | 71.1, | 70.8, | 70.9, | 63.6, | 66.8, | 64.1, | 56.8, | 50.2, |
| | Mann/50 mM Tre | 93.3 | 93.9 | 93.9 | 93.2 | 91.8 | 92.6 | 90.5 | 88.5 | 79.3 | 57.8 |
| | (429 mOsm/kg) | | | | | | | | | | |
| | 100 mM NaCl/125 mM | 53.4, | 55.3, | 58.7, | 55.8, | 56.2, | 49.9, | 54.3, | 56.1, | 53.0, | 53.3, |
| | Mann/50 mM Tre | 93.3 | 93.9 | 94.2 | 93.5 | 92.4 | 92.3 | 89.6 | 90.8 | 86.4 | 68.3 |
| | (536 mOsm/kg) | | | | | | | | | | |
| | 50 mM NaCl/50 mM | 51.5, | 54.4, | 53.6, | 53.3, | 53.8, | 51.0, | 51.3, | 54.6, | 51.7, | 56.4, |
| | Mann/125 mM Tre | 93.4 | 94.0 | 94.2 | 93.4 | 92.4 | 93.5 | 90.8 | 91.7 | 87.5 | 71.8 |
| | (380 mOsm/kg) | | | | | | | | | | |
| | 100 mM NaCl/50 mM | 51.7, | 51.8, | 55.4, | 46.0, | 54.5, | 52.7, | 52.2, | 54.8, | 52.2, | 56.9, |
| | Mann/125 mM Tre | 93.4 | 94.3 | 94.3 | 93.8 | 91.8 | 93.2 | 90.2 | 91.5 | 86.8 | 69.7 |
| | (541 mOsm/kg) | | | | | | | | | | |
| | 50 mM NaCl/125 mM | 51.3, | 52.8, | 54.8, | 52.5, | 54.5, | 52.7, | 52.9, | 54.1, | 50.7, | 62.0, |
| | Mann/125 mM Tre | 93.7 | 94.2 | 84.3 | 93.9 | 92.7 | 93.2 | 90.8 | 91.8 | 87.4 | 71.3 |
| | (568 mOsm/kg) | | | | | | | | | | |
| | 100 mM NaCl/125 mM | 54.4, | 56.7, | 60.8, | 60.2, | 60.2, | 57.6, | 58.1, | 57.8, | 54.2, | 63.3, |
| | Mann/125 mM Tre | 93.6 | 93.8 | 94.6 | 93.6 | 92.5 | 93.6 | 90.6 | 91.3 | 87.2 | 70.1 |
| | (693 mOsm/kg) | | | | | | | | | | |

*denotes insufficient sample for analysis

ANOVA results showed no significant effects due to the various excipient combinations upon storage after 70 days at 5° C. by IE-HPLC method. Following storage at 25° C. and 32° C., low concentrations of sodium chloride (50 mM) and high concentrations of trehalose (125 mM) provide significantly more stable conditions (greater purity) than the converse.

### Conclusion

Overall, TV-1106 was quite stable in all of the excipient combinations following storage at 5° C. for up to 70 days. Changes in sample purity were between 0.5 and 1.0% as determined by RP-HPLC, less than 0.5% as determined by SEC-HPLC (assuming t=0 purity was 99.4%—the value of bulk TV-1106 and not the reported t=0 value in which there were issues with sample preparation), and at most 0.1% as determined by IE-HPLC. Larger decreases in purity were observed following incubation at 25° C. and 32° C. over a smaller time frame. TV-1106 sample recovery, as determined by HPLC, was near 100% for samples stored at 5° C.; however at elevated temperatures large increases in recovery were observed likely due to evaporation, although this was not investigated further.

ANOVA results showed no significant effects due to the various excipient combinations upon storage after 70 days at 5° C. by any of the HPLC methods. Optimization of the formulation composition to a target physiologically relevant osmolality (300 mOsm/kg) by minimizing the amount of each excipient in each formulation provides a formulation comprising 10 mM histidine, pH 6.5, 50 mM NaCl, 50 mM mannitol and 80-100 mM trehalose. The estimated osmolality of these solutions is on the order of 350 mOsm/kg.

### Example 2

### Stability Study at Different pH Values

Preformulation studies demonstrated pH as one of the critical factors that impact product quality attributes. To confirm the pH range of lead TV-1106 formulations, a 2-week stability study of 100 TV-1106 formulations consisting of 20 mM histidine, 100 mM NaCl, 75 mM trehalose and 20 mM phosphate, 180 mM mannitol, 60 mM trehalose at pH 5.5, 6.0, 6.5, and 7.0 was conducted with compendial grade excipients under stress condition.

### Sample Preparation

TV-1106 was received from Teva Biopharmaceuticals at a protein concentration of 31.1 mg/mL prepared in 20 mM histidine, 160 mM mannitol, and 75 mM trehalose, pH 6.5 buffer. A volume of 6 mL of the TV-1106, was dialyzed against 2 liter (500 mL×4 changes) of the corresponding test buffered solutions, as shown in Tables 17a and 17b, at 4° C. for 24 hours. The recovered protein solution was concentrated to approximately 50 and 100 mg/mL using centrifugation in Pierce® Concentrators, 20K MWCO (p/n 89886A). Final protein concentration was determined by A280 using an extinction coefficient of 0.6 O.D.=1 mg/mL TV-1106.

### Incubation Conditions

A volume of 0.4 mL of TV-1106 bulk drug solutions were placed in 3 mL flint vials from Schott (6800-0316) and subsequently stoppered and capped. The upright vials were then stored at 32° C.

### RP-, SEC- and IE-HPLC Analysis

RP-, SEC-, and EE-HPLC analysis was carried out immediately following sample preparation and after incubation at the appropriate stress condition.

### Sample Appearance and pH

Sample appearance and pH determination were carried out immediately following sample preparation and after incubation at the appropriate stress condition

### Results

### pH and A280 Readings

Following a two week incubation at 32° C. all test samples remained visually clear and free of precipitate, regardless of storage conditions. Protein concentration as determined from the A280 and sample pH values did not differ significantly from their original values following the two week period (Tables 10a and 10b, and 11a and 11b, respectively).

TABLE 10a
2-week pH stability of TV-1106: TV-1106 concentration
determined from A280 nm values

| | | | TV-1106 (mg/mL)* | | | |
|---|---|---|---|---|---|---|
| | | | | 32° C. incubation | | |
| | | | | t = 3 | t = 7 | t = 14 |
| Sample Composition | | | t = 0 | day | day | day |
| 4151a- 20 mM | 50 | pH 7.0 | 47.2 | 46.0 | 52.6 | 54.3 |
| Histidine, | mg/mL | pH 6.5 | 46.4 | 50.8 | 50.1 | 48.3 |
| 100 mM NaCl, | TV-1106 | pH 6.0 | 47.8 | 49.3 | 50.7 | 56.3 |
| 75 mM Trehalose | | pH 5.5 | 48.4 | 44.8 | 50.3 | 53.8 |
| (0.07% Tween-80) | 100 | pH 7.0 | 101.2 | 103.3 | 100.5 | 108.0 |
| | mg/mL | pH 6.5 | 96.8 | 89.3 | 93.2 | 100.5 |
| | TV-1106 | pH 6.0 | 99.0 | 92.0 | 99.2 | 109.0 |
| | | pH 5.5 | 100.7 | 104.7 | 107.8 | 110.2 |

*TV-1106 concentration determined from A280 nm of 0.6 = 1.0 mg/mL TV-1106.

TABLE 10b
2-week pH stability of TV-1106: TV-1106 concentration
determined from A280 nm values

| | | | TV-1106 (mg/mL)* | | | |
|---|---|---|---|---|---|---|
| | | | | 32° C. incubation | | |
| | | | | t = 3 | t = 7 | t = 14 |
| Sample Composition | | | t = 0 | day | day | day |
| TBU-20 mM Phosphate, | 50 | pH 7.0 | 51.8 | 49.4 | 45.3 | 48.1 |
| 180 mM Mannitol, | mg/mL | pH 6.5 | 47.8 | 46.4 | 44.4 | 45.3 |
| 60 mM Trehalose | TV-1106 | pH 6.0 | 48.5 | 41.9 | 46.8 | 42.5 |
| | | pH 5.5 | 46.8 | 44.2 | 48.5 | 51.9 |
| | 100 | pH 7.0 | 92.7 | 91.8 | 89.7 | 90.8 |
| | mg/mL | pH 6.5 | 80.8 | 76.3 | 85.5 | 88.0 |
| | TV-1106 | pH 6.0 | 84.7 | 79.5 | 90.5 | 82.5 |
| | | pH 5.5 | 77.0 | 76.3 | 78.3 | 73.5 |

| | | | | | |
|---|---|---|---|---|---|
| 4132a- 20 mM Phos, 160 Mannitol, | 50 mg/mL | 48.4 | 42.0 | 42.8 | 43.3 |
| 50 mM Trehalose, pH 6.5 | 100 mg/mL | 95.7 | 84.7 | 82.0 | 99.5 |
| 2P- 10 mM Phos, 2000 Mannitol, | 50 mg/mL | 47.6 | 43.3 | 47.1 | 47.4 |
| 60 mM Trehalose, pH 7.2 | 100 mg/mL | 84.8 | 91.8 | 97.5 | 100.5 |

*TV-1106 concentration determined from A280 nm of 0.6 = 1.0 mg/mL TV-1106.

TABLE 11a
.2-week pH stability of TV-1106: pH values

| | | | Sample pH | | | |
|---|---|---|---|---|---|---|
| | | | | 32° C. incubation | | |
| | | | | t = 3 | t = 7 | t = 14 |
| Sample Composition | | | t = 0 | day | day | day |
| 4151a- 20 mM | 50 | pH 7.0 | 7.1 | 6.9 | 7.1 | 6.9 |
| Histidine, | mg/mL | pH 6.5 | 6.6 | 6.6 | 6.6 | 6.5 |
| 100 mM NaCl, | TV-1106 | pH 6.0 | 6.2 | 6.1 | 6.1 | 6.1 |
| 75 mM Trehalose | | pH 5.5 | 5.7 | 5.7 | 5.6 | 5.6 |
| (0.07% Tween-80) | 100 | pH 7.0 | 6.9 | 6.9 | 7.1 | 6.8 |
| | mg/mL | pH 6.5 | 6.6 | 6.6 | 6.6 | 6.5 |
| | TV-1106 | pH 6.0 | 6.2 | 6.1 | 6.1 | 6.1 |
| | | pH 5.5 | 5.7 | 5.7 | 5.7 | 5.6 |

TABLE 11b
.2-week pH stability of TV-1106: pH values

| | | | Sample pH | | | |
|---|---|---|---|---|---|---|
| | | | | 32° C. incubation | | |
| | | | | t = 3 | t = 7 | t = 14 |
| Sample Composition | | | t = 0 | day | day | day |
| TBU-20 mM Phosphate, | 50 | pH 7.0 | 6.9 | 6.9 | 6.9 | 6.8 |
| 180 mM Mannitol, | mg/mL | pH 6.5 | 6.4 | 6.4 | 6.5 | 6.4 |
| 60 mM Trehalose | TV-1106 | pH 6.0 | 6.0 | 6.0 | 6.1 | 6.0 |
| | | pH 5.5 | 5.8 | 5.8 | 5.9 | 5.8 |
| | 100 | pH 7.0 | 6.7 | 6.7 | 6.8 | 6.7 |
| | mg/mL | pH 6.5 | 6.3 | 6.3 | 6.4 | 6.3 |
| | TV-1106 | pH 6.0 | 6.0 | 5.9 | 6.0 | 5.9 |
| | | pH 5.5 | 5.8 | 5.8 | 5.8 | 5.8 |

| | | | | | |
|---|---|---|---|---|---|
| 4132a- 20 mM Phos, 160 Mannitol, | 50 mg/mL | 6.5 | 6.4 | 6.4 | 6.4 |
| 50 mM Trehalose, pH 6.5 | 100 mg/mL | 6.4 | 6.3 | 6.4 | 6.3 |
| 2P- 10 mM Phos, 2000 Mannitol, | 50 mg/mL | 7.0 | 6.9 | 6.9 | 6.9 |
| 60 mM Trehalose, pH 7.2 | 100 mg/mL | 7.0 | 6.8 | 6.8 | 6.7 |

### EFFECT of pH on HPLC Purity and Recovery

Sample recovery and purity was evaluated by RP-HPLC (Tables 12a and 12b), SEC-HPLC (Tables 13a and 13b), and EE-HPLC (Tables 14a and 14b).

TABLE 12a
2-week pH stability of TV-1106: RP-HPLC results

| | | | TV-1106 conc. (mg/mL), % purity | | | |
|---|---|---|---|---|---|---|
| | | | | 32° C. incubation | | |
| | | | | t = 3 | t = 7 | t = 14 |
| Sample Composition | | | t = 0 | day | day | day |
| 4151a- 20 mM | 50 | pH 7.0 | 51.4, 91.3 | 49.1, 90.1 | 49.5, 88.3 | 48.6, 86.5 |
| Histidine, | mg/mL | pH 6.5 | 52.9, 91.2 | 45.5, 89.9 | 46.7, 88.0 | 46.2, 86.5 |
| 100 mM NaCl, | TV-1106 | pH 6.0 | 51.9, 91.4 | 49.5, 89.1 | 47.4, 86.4 | 44.2, 85.1 |
| 75 mM Trehalose | | pH 5.5 | 52.3, 91.0 | 44.8, 88.4 | 43.8, 82.7 | 44.2, 80.7 |
| (0.07% Tween-80) | 100 | pH 7.0 | 98.1, 90.9 | 100.4, 89.8 | 96.2, 88.3 | 92.6, 86.7 |
| | mg/mL | pH 6.5 | 91.7, 91.1 | 87.6, 89.6 | 87.4, 87.6 | 85.1, 86.7 |
| | TV-1106 | pH 6.0 | 94.0, 91.0 | 88.0, 89.1 | 89.4, 86.6 | 82.8, 85.2 |
| | | pH 5.5 | 114.2, 90.8 | 102.1, 87.5 | 94.6, 83.7 | 85.2, 80.6 |

TABLE 12b
2-week pH stability of TV-1106: RP-HPLC results

| | | | TV-1106 conc. (mg/mL), % purity | | | |
|---|---|---|---|---|---|---|
| | | | | 32° C. incubation | | |
| | | | | t = 3 | t = 7 | t = 14 |
| Sample Composition | | | t = 0 | day | day | day |
| TBU-20 mM Phosphate, | 50 | pH 7.0 | 51.2, 91.3 | 48.4, 90.1 | 46.4, 89.3 | 48.6, 87.7 |
| 180 mM Mannitol, | mg/mL | pH 6.5 | 50.5, 91.2 | 51.4, 89.5 | 42.5, 88.7 | 46.2, 87.4 |
| 60 mM Trehalose | TV-1106 | pH 6.0 | 49.0, 91.4 | 41.2, 88.8 | 44.6, 87.3 | 44.2, 85.4 |
| | | pH 5.5 | 50.0, 91.0 | 43.0, 88.2 | 46.5, 85.7 | 44.2, 85.1 |
| | 100 | pH 7.0 | 115.2, 90.9 | 89.4, 90.0 | 90.3, 88.6 | 92.6, 87.2 |
| | mg/mL | pH 6.5 | 84.1, 91.1 | 73.4, 89.5 | 82.6, 87.6 | 85.1, 86.6 |
| | TV-1106 | pH 6.0 | 84.2, 91.0 | 75.7, 88.0 | 85.4, 86.2 | 82.8, 84.5 |
| | | pH 5.5 | 118.1, 90.8 | 73.1, 87.7 | 74.3, 85.2 | 85.2, 84.3 |

| | | | | | |
|---|---|---|---|---|---|
| 4132a- 20 mM Phos, 160 Mannitol, | 50 mg/mL | 49.7, 91.5 | 42.0, 89.6 | 42.6, 88.5 | 39.2, 88.7 |
| 50 mM Trehalose, pH 6.5 | 100 mg/mL | 111.8, 90.8 | 83.0, 89.3 | 78.6, 88.0 | 79.0, 86.5 |
| 2P- 10 mM Phos, 200 Mannitol, | 50 mg/mL | 50.3, 90.8 | 42.2, 89.7 | 44.5, 89.0 | 44.1, 87.8 |
| 60 mM Trehalose, pH 7.2 | 100 mg/mL | 113.7, 90.8 | 87.9, 89.7 | 88.4, 88.2 | 89.4, 86.8 |

TABLE 13a
2-week pH stability of TV-1106: SEC-HPLC results

| | | | TV-1106 conc. (mg/mL), % purity | | | |
|---|---|---|---|---|---|---|
| | | | | 32° C. incubation | | |
| | | | | t = 3 | t = 7 | t = 14 |
| Sample Composition | | | t = 0 | day | day | day |
| 4151a- 20 mM | 50 | pH 7.0 | 52.5, 98.9 | 51.4, 98.9 | 53.6, 98.8 | 53.9, 98.8 |
| Histidine, | mg/mL | pH 6.5 | 58.8, 98.9 | 46.7, 98.7 | 51.8, 98.9 | 52.0, 98.7 |
| 100 mM NaCl, | TV-1106 | pH 6.0 | 53.6, 98.9 | 50.6, 98.8 | 52.1, 98.7 | 49.6, 98.6 |
| 75 mM Trehalose | | pH 5.5 | 53.3, 98.8 | 45.6, 98.5 | 51.4, 98.5 | 51.4, 98.2 |
| (0.07% Tween-80) | 100 | pH 7.0 | 107.6, 98.9 | 102.5, 98.7 | 105.1, 98.6 | 102.1, 98.4 |
| | mg/mL | pH 6.5 | 101.1, 98.8 | 91.5, 98.7 | 94.6, 98.6 | 91.2, 98.4 |
| | TV-1106 | pH 6.0 | 101.7, 98.7 | 93.9, 98.6 | 97.8, 98.4 | 87.5, 98.3 |
| | | pH 5.5 | 111.6, 98.7 | 106.9, 98.4 | 106.1, 98.1 | 104.5, 97.8 |

TABLE 13b
2-week pH stability of TV-1106: SEC-HPLC results

| | | | TV-1106 conc. (mg/mL), % purity | | | |
|---|---|---|---|---|---|---|
| | | | | 32° C. incubation | | |
| | | | | t = 3 | t = 7 | t = 14 |
| Sample Composition | | | t = 0 | day | day | day |
| TBU-20 mM Phosphate, | 50 | pH 7.0 | 53.2, 98.8 | 49.9, 98.8 | 48.5, 98.9 | 49.9, 98.7 |
| 180 mM Mannitol, | mg/mL | pH 6.5 | 52.6, 98.7 | 54.4, 98.7 | 44.7, 98.7 | 45.6, 98.6 |
| 60 mM Trehalose | TV-1106 | pH 6.0 | 51.2, 98.6 | 42.4, 98.3 | 46.9, 98.4 | 45.4, 98.1 |
| | | pH 5.5 | 52.2, 98.4 | 44.7, 98.0 | 49.8, 98.2 | 45.9, 98.0 |
| | 100 | pH 7.0 | 96.6, 98.7 | 94.9, 98.3 | 94.7, 98.3 | 86.6, 98.2 |
| | mg/mL | pH 6.5 | 87.6, 98.6 | 76.3, 98.2 | 86.8, 98.2 | 82.3, 98.1 |
| | TV-1106 | pH 6.0 | 86.0, 99.2 | 78.6, 97.9 | 91.1, 97.9 | 83.2, 97.7 |
| | | pH 5.5 | 82.3, 98.4 | 75.9, 97.8 | 82.3, 97.8 | 76.1, 97.4 |

| | | | | | |
|---|---|---|---|---|---|
| 4132a- 20 mM Phos, 160 Mannitol, | 50 mg/mL | 51.2, 98.8 | 43.2, 98.7 | 44.3, 98.7 | 41.9, 98.6 |
| 50 mM Trehalose, pH 6.5 | 100 mg/mL | 89.8, 98.7 | 87.5, 98.3 | 83.7, 98.2 | 86.1, 98.1 |
| 2P- 10 mM Phos, 200 Mannitol, | 50 mg/mL | 52.1, 98.6 | 44.7, 98.6 | 46.6, 98.6 | 46.9, 98.7 |
| 60 mM Trehalose, pH 7.2 | 100 mg/mL | 101.6, 98.5 | 91.5, 98.1 | 97.4, 98.1 | 94.1, 98.0 |

TABLE 14a
2-week pH stability of TV-1106: IE-HPLC results

| | | | TV-1106 conc. (mg/mL), % purity | | | |
|---|---|---|---|---|---|---|
| | | | | 32° C. incubation | | |
| | | | | t = 3 | t = 7 | t = 14 |
| Sample Composition | | | t = 0 | day | day | day |
| 4151a- 20 mM | 50 | pH 7.0 | 54.4, 93.0 | 49.7, 88.8 | 46.3, 80.8 | 38.5, 69.5 |
| Histidine, | mg/mL | pH 6.5 | 58.2, 93.5 | 46.9, 89.8 | 47.5, 86.0 | 38.2, 78.0 |
| 100 mM NaCl, | TV-1106 | pH 6.0 | 54.0, 93.5 | 51.8, 91.4 | 48.4, 88.4 | 41.3, 78.1 |
| 75 mM Trehalose | | pH 5.5 | 55.1, 93.7 | 45.9, 91.8 | 45.8, 83.5 | 39.9, 76.4 |
| (0.07% Tween-80) | 100 | pH 7.0 | 113.5, 93.3 | 98.2, 87.4 | 93.5, 81.8 | 76.1, 70.8 |
| | mg/mL | pH 6.5 | 107.8, 93.4 | 88.9, 90.3 | 87.3, 85.3 | 83.9, 78.1 |
| | TV-1106 | pH 6.0 | 106.5, 92.5 | 91.5, 91.8 | 92.1, 88.3 | 81.0, 79.1 |
| | | pH 5.5 | 115.6, 94.0 | 106.1, 92.6 | 95.6, 84.0 | 90.2, 77.1 |

TABLE 14b
2-week pH stability of TV-1106: IE-HPLC results

| | | | TV-1106 conc. (mg/mL), % purity | | | |
|---|---|---|---|---|---|---|
| | | | | 32° C. incubation | | |
| | | | | t = 3 | t = 7 | t = 14 |
| Sample Composition | | | t = 0 | day | day | day |
| TBU-20 mM Phosphate, | 50 | pH 7.0 | 56.7, 93.5 | 48.2, 89.2 | 43.5, 82.9 | 38.1, 72.1 |
| 180 mM Mannitol, | mg/mL | pH 6.5 | 52.7, 93.4 | 51.9, 90.4 | 42.3, 86.8 | 39.9, 81.5 |
| 60 mM Trehalose | TV-1106 | pH 6.0 | 54.4, 92.5 | 43.2, 92.0 | 45.8, 89.9 | 36.0, 802 |
| | | pH 5.5 | 54.5, 93.8 | 44.0, 92.6 | 46.8, 85.9 | 40.2, 81.9 |
| | 100 | pH 7.0 | 103.0, 93.0 | 93.2, 89.0 | 86.1, 83.5 | 71.6, 74.7 |
| | mg/mL | pH 6.5 | 92.8, 92.2 | 73.7, 90.5 | 82.7, 87.5 | 79.6, 81.4 |
| | TV-1106 | pH 6.0 | 92.0, 93.8 | 78.0, 91.3 | 88.6, 89.8 | 75.0, 80.2 |
| | | pH 5.5 | 86.1, 92.7 | 75.8, 91.4 | 75.4, 85.5 | 72.7, 82.0 |

| | | | | | |
|---|---|---|---|---|---|
| 4132a- 20 mM Phos, 160 Mannitol, | 50 mg/mL | 53.4, 93.7 | 43.3, 90.7 | 42.4, 90.7 | 35.8, 80.5 |
| 50 mM Trehalose, pH 6.5 | 100 mg/mL | 97.6, 93.3 | 86.1, 89.9 | 77.9, 87.4 | 78.2, 82.1 |
| 2P- 10 mM Phos, 200 Mannitol, | 50 mg/mL | 54.7, 92.1 | 42.6, 87.2 | 42.9, 83.3 | 32.2, 73.6 |
| 60 mM Trehalose, pH 7.2 | 100 mg/mL | 110.3, 93.1 | 89.3, 88.7 | 902, 85.1 | 80.8, 76.8 |

### Conclusion

Following two weeks of storage all test samples remained visually clear and free of precipitate. Sample pH remained near (+0.1) the original experimental value throughout the course of the experiment. Decrease in sample purity as determined by RP-HPLC analysis was near 5% at 32° C. after two weeks of storage for samples prepared in the range of pH 6.5-7.0. Sample purity as determined by SEC-HPLC analysis was near 0.1% following two weeks of storage at 32° C. for test samples prepared in the range of pH 6.5-7.0. Decrease in sample purity as determined by IE-HPLC analysis was between 10 and 15% after two weeks of storage 32° C. for test samples prepared in the range of pH 5.5-6.5.

### Example 3

### Identification of Asparagine Deamidation and Aspartate Isomerization Sites in TV-1106

The forced degradation studies of Example 1 had suggested that aspartate isomerization and/or asparagine deamidation occurs at multiple residues of TV-1106. Studies were undertaken to identify and characterize the main susceptible sites of asparagine deamidation and aspartate isomerization in TV-1106 under liquid formulation conditions.

### Size Exclusion HPLC (SE-HPLC)

SEC was used to detect and quantify aggregates in TV-1106. Aggregates, dimer and monomer of TV-1106 were separated by a TosoHaas G3000SWXL column (7.8 mm×30 cm).

### Reversed Phase HPLC (RP-HPLC)

TV-1106 samples were analyzed by RP-HPLC using an Agilent ZORBAC® SB-C8 column.

Imaging Capillary Iso-Electrofocusing (iCIEF)

The cIEF method was developed on a Convergent Biosciences iCE 280 instrument. The method conditions are listed in Table 15.

TABLE 15

| | Conditions |
|---|---|
| Assay solution composition | 0.54 mg/mL sample |
| | 3.70M urea |
| | 0.31% methyl cellulose |
| | 2.78% Pharmalytes pH |
| | 0.93% Pharmalytes pH |
| | 0.2% pI marker 5.12 |
| | 0.2% pI marker 7.05 |
| Focusing period 1 | 1 minute, 1500 V |
| Focusing period 2 | 10 minutes, 3000 V |
| Buffer injection | 225 sec, 2000 mbar |
| Sample Injection | 150 sec, 2000 mbar |
| Autosampler Temperature | 10° C. |
| Absorbance detection | 280 nm |

### Lys-C Peptide Mapping

Protein samples were reduced in a solution of 6N Guanidine HCl and 50 mM DTT pH 7.5 with 2 to 5 mg/mL protein concentration by incubating at room temperature for 30 minutes. After reduction LAM was added to achieve approximately 15 mM concentration. Alkylation of the sulfhydryl groups was achieved by incubation in dark at room temperature for 60 minutes. After alkylation the residual LAM were quenched by addition of equimolar DTT.

The reduced and alkylated samples were buffer exchanged into 4M urea solution buffered at pH 7.0. Recombinant Lys-C was added to achieve a 1:10 enzyme to protein ratio. Digestion was carried out at 37° C. for 3 hours.

After completion of digestion the sample was quenched by addition of an equal volume of 8M Guanidine HCl. The resulting peptide fragments were analyzed by RP-HPLC using an ultra high resolution column installed on a UHPLC system.

### Lys-C/Trypsin Peptide Mapping

The reduction and alkylation procedure was the same as described above for the Lys-C peptide map method. After reduction and alkylation the sample was buffer exchanged into 2M urea buffered at pH 7.5. Lys-C/Trypsin enzyme mix was added at an enzyme protein ratio of 1:10. The solution was incubated at 37° C. for 3 hours. The sample was diluted 1:2 and continued incubation at 37° C. for an additional 4 hours. After completion of digestion the sample was quenched by the addition of 5 μL of 5% TFA solution.

### Results

### Detection of Charge Isoforms of TV-1106

During liquid formulation development the lead formulations underwent accelerated stability studies to estimate purity profile of the product during the intended shelf life. Projected purity during 24 month at 2-8° C. of liquid formulated TV-1106 by SE-HPLC, RP-HPLC, iCIEF and ISOQUANT® methods had been obtained.

The purity as measured by SE-HPLC and RP-HPLC is relatively stable under these conditions, which in turn implies that no significant levels of aggregation and oxidative degradation occur. However, the purity measured by iCIEF shows a significant decrease during the intended shelf life and ISOQUANT® assay detected increased amounts of isoaspartate. This implies that significant levels of charge based isoforms are generated most likely due to deamidation of asparagine residues and/or isomerization of aspartate residues. No formulations were found which would eliminate the formation at these levels of charge based variants. Consequently, it became essential to understand fully underlying reasons for increased impurity levels, such as the type of modification, site of modification, and extent of modification.

TABLE 16
Projected purity during 24 month at 2-8° C. of liquid
formulated TV-1106 by different analytical methods

| | | | | RP- | | |
|---|---|---|---|---|---|---|
| | SE-HPLC | | | HLPC | | |
| | Agg./Dimer/MP³ | | | MP³ | iCIEF | |
| | T0: | | | T0: | MP³ | IsoQuant |
| Formulation | ≦0.1%/~0.3%/~99% | | | ~93% | T0: ~84% | T0: ~1% |
| 100-PMT-c¹ | 0.4% | 1.3% | 97.8% | 87.2% | 70.5% | 11.2% |
| 100-PMT-v² | 0.3% | 1.2% | 98.1% | 86.6% | 73.3% | 8.0% |
| 50-PMT-c¹ | 0.2% | 0.9% | 98.4% | 87.4% | 69.6% | 9.2% |
| 2P DP-v² | 0.6% | 0.7% | 98.3% | 90.1% | etc. 73% | 7.0% |
| 50-HN-c¹ | 0.1% | 0.6% | 98.9% | 89.8% | 59.3% | 23.1% |
| 50-HN-v² | 0.1% | 0.5% | 99.0% | 88.4% | 61.1% | 24.5% |
| 50-HNMT-c¹ | 0.1% | 0.7% | 98.8% | 89.5% | 61.7% | 23.7% |
| 50-HNMT-v² | 0.1% | 0.6% | 98.8% | 87.9% | 61.6% | 22.0% |

¹Cartridge;
²Vial;
³Main Peak

### Lyc-C Peptide Map for Detection of Main Variants

Table 17 shows the assignments of the peaks in a peptide map to peptide fragments from the Lys-C digestion of TV1106 based on accurate mass MS analysis. As seen from Table 17, 98.1% coverage of the amino acid sequence of TV-1106 was achieved. Most peptide peaks were chromatographically well resolved and only limited numbers of minor fragments were produced in the procedure.

TABLE 17
Sequence coverage of Lyc-C digest of TV-1106

| Peptide | | Theoretical | Observed | | Sequence | Missed |
|---|---|---|---|---|---|---|
| ID | RT | mass | mass | Peak area | Location | cleavage |
| L2 | 16.682 | 972.5141 | 972.5174 | 13496861 | A(5-12) | 0 |
| L3 | 22.216 | 950.4345 | 950.4379 | 11622813 | A(13-20) | 0 |
| L4 | 113.828 | 2489.2777 | 2489.2922 | 62846600 | A(21-41) | 0 |
| L5 | 43.397 | 1148.6077 | 1148.6127 | 33529400 | A(42-51) | 0 |
| L6 | 14.202 | 1497.5712 | 1497.5753 | 8786267 | A(52-64) | 0 |
| L7 | 47.152 | 1016.5291 | 1016.5336 | 34536492 | A(65-73) | 0 |
| L8 | 56.7 | 2348.0269 | 2348.036 | 34461172 | A(74-93) | 0 |
| L9 | 21.978 | 1713.7893 | 1713.7955 | 24358562 | A(94-106) | 0 |
| L10 | 85.366 | 3570.6872 | 3570.7024 | 47020632 | A(107-136) | 0 |
| L12 | 105.017 | 2806.4635 | 2806.4792 | 50850112 | A(138-159) | 0 |
| L11-12 | 97.25 | 2934.5585 | 2934.5722 | 1465075 | A(137-159) | 1 |
| L13 | 11.193 | 465.27 | 465.27 | 2680226 | A(160-162) | 0 |
| L14 | 17.128 | 1370.5595 | 1370.3638 | 14952766 | A(163-174) | 0 |
| L15 | 30.984 | 771.4313 | 771.4327 | 19917696 | A(175-181) | 0 |
| L16 | 17.112 | 1073.5353 | 1073.539 | 18881250 | A(182-190) | 0 |
| L18 | 13.332 | 543.3493 | 543.3499 | 4618501 | A(196-199) | 0 |
| L19 | 14.061 | 705.348 | 705.3491 | 7420247 | A(200-205) | 0 |
| L20 | 23.058 | 853.4446 | 853.4476 | 19619342 | A(206-212) | 0 |
| L21 | 59.607 | 1528.8627 | 1528.8705 | 37967964 | A(213-225) | 0 |
| L22 | 19.817 | 879.4338 | 879.437 | 13528091 | A(226-233) | 0 |
| L23 | 24.422 | 788.4644 | 788.4661 | 20392436 | A(234-240) | 0 |
| L24 | 41.813 | 2584.1105 | 2584.1203 | 45335336 | A(241-262) | 0 |
| L25 | 17.071 | 1442.6348 | 1442.6398 | 12594516 | A(263-274) | 0 |
| L26 | 10.831 | 259.1896 | 259.1895 | 1933104 | A(275-276) | 0 |
| L27-28 | 22.103 | 1304.6105 | 1304.6155 | 21872158 | A(277-286) | 1 |
| L29 | 88.595 | 2973.3372 | 2973.3553 | 37899616 | A(287-313) | 0 |
| L30 | 10.849 | 520.2315 | 520.2317 | 3107963 | A(314-317) | 0 |
| L31 | 13.236 | 694.3286 | 694.3294 | 3262150 | A(318-323) | 0 |
| L32 | 118.782 | 3383.8217 | 3383.8407 | 69745016 | A(324-351) | 0 |
| L33 | 18.254 | 983.4811 | 983.4142 | 13087114 | A(352-359) | 0 |
| L34 | 15.29 | 1551.5905 | 1551.5947 | 9024221 | A(360-372) | 0 |
| L35-36 | 76.407 | 2044.0881 | 2044.0971 | 44884124 | A(373-389) | 1 |
| L37 | 59.912 | 1656.7454 | 1656.7527 | 19444956 | A(390-402) | 0 |
| L38 | 53.257 | 1351.7612 | 1351.7689 | 22388120 | A(403-413) | 0 |
| L39-40 | 62.431 | 2051.1739 | 2051.1844 | 65855204 | A(414-432) | I |
| L41 | 6.328 | 389.2274 | 389.2273 | 2473744 | A(433-436) | 0 |
| L42-43 | 12.347 | 1028.4532 | 1028.4542 | 6446612 | A(437-444) | 1 |
| L44 | 108.397 | 2673.3077 | 2673.3209 | 30508568 | A(445-466) | 0 |
| L45 | 15.57 | 1001.5506 | 1001.5539 | 11796763 | A(467-475) | 0 |
| L46 | 65.088 | 3029.4045 | 3029.416 | 27923610 | A(476-500) | 0 |
| L47 | 70.979 | 2259.0154 | 2259.0244 | 32192694 | A(501-519) | 0 |
| L48 | 13.418 | 672.3919 | 672.3925 | 4470525 | A(520-524) | 0 |
| L48-49 | 13.125 | 800.4868 | 800.4874 | 2958577 | A(520-S25) | 1 |
| L50 | 54.152 | 999.5964 | 999.6018 | 16509056 | A(526-534) | 0 |
| L51-52 | 7.141 | 508.3122 | 508.3116 | 924021 | A(535-538) | 1 |
| L51-53 | 113.889 | 808.4919 | 808.4843 | 199486 | A(535-541) | 2 |
| L54 | 13.434 | 516.2908 | 516.2915 | 3840620 | A(542-545) | 0 |
| L55 | 71.754 | 1341.6275 | 1341.6334 | 33406282 | A(546-557) | 0 |
| L58 | 16.569 | 1069.4386 | 1069.4409 | 8907826 | A(565-573) | 0 |
| L57-58 | 16.869 | 1498.6246 | 1498.6539 | 84793 | A(561-573) | 1 |
| L59-60 | 123.632 | 5700.6079 | 5700.0318 | 1537798 | A(574-623) | 1 |
| L62 | 70.103 | 3415.5991 | 3415.6117 | 43086604 | A(627-655) | 0 |
| L63 | 133.189 | 5122.7747 | 5122.8006 | 24652234 | A(656-700) | 0 |
| L64 | 96.648 | 2789.4018 | 2789.4163 | 23489894 | A(701-725) | 0 |
| L65 | 11.517 | 625.3071 | 625.3072 | 2672196 | A(726-730) | 0 |
| L66 | 31.436 | 1488.6845 | 1488.6889 | 32711720 | A(731-743) | 0 |
| L67 | 60.401 | 1332.6649 | 1332.6717 | 30777892 | A(744-753) | 0 |
| L68 | 10.868 | 507.1999 | 507.1999 | 2425136 | A(754-757) | 0 |

One strategy used to search for susceptible sites was to analyze samples that underwent various degrees of stress conditions and compare the resulting peptide maps. This approach led to the identification of two peptide fragments, L64 and L66, which showed significant and increased levels of modifications under increasingly severe stress conditions.

The abundance of the chromatographic peak corresponding to L64 peptide decreased with increasingly severe stress conditions while at the same time the abundance of another peak was increasing. The mass spectra corresponding to the unmodified and modified L66 peaks showed that the two mass spectra are identical indicating that the modification did not result in a change of the molecular weight. It is most likely that isomerization of an aspartate residue is involved in the modification.

The abundance of the chromatographic peak corresponding to L66 peptide decreased with increasingly severe stress conditions while at the same time the abundance of some other peaks were increasing. The mass spectra corresponding to the unmodified and modified L66 peaks showed a one Dalton difference in molecular weight. The accurate mass difference closely matches that corresponding to the deamidation of an asparagine residue.

Lys-C Peptide Map with LC/MS/MS for Identification of Deamidation Site for Deamidated Variant of TV-1106

Lys-C peptide L66 has two potential deamidation sites; asparagine residues at 734 and 737 In order to elucidate the location of the deamidation MS/MS experiments were carried out on the unmodified and the deamidated peptides using the m/z 745.45 and 745.84 ions, respectively, as precursors for fragmentations.

MS/MS spectra obtained from the L66 and deamiated L66 peptides provided evidence that the site of deamidation is the N734 residue. Its mass increased by one Dalton while the masses of all the subsequent residues remained the same. Therefore, it was concluded that the susceptible site revealed by the observations regarding the L66 peptide is N734.

Lys-C/Trypsin Peptide Map with LC/MS for Identification of Isomerization Site for Isomerized Variant of TV-1106

Peptide map chromatograms for three TV-1106 samples exposed to different degrees of forced degradation conditions showed that the abundance of the chromatographic peak corresponding to L64 peptide decreased with increasingly severe stress conditions while at the same time the abundance of another peak increased. The mass spectra corresponding to the unmodified and modified L64 peaks were identical indicating that the modification did not result in a change of the molecular weight. It was concluded that the susceptible site revealed by the observations regarding the L64 peptide is D715.

### Conclusion

The peptide map method developed achieved >98% sequence coverage, had no method induced modifications, and had the capability to chromatographically resolve modified peptides. The method was utilized to study TV-1106 materials that were exposed to various degrees of stress conditions. Two main susceptible sites were identified, D715 which underwent isomerization at enhanced rate in liquid formulation; and N734 that underwent deamidation. Both of these sites are within the growth hormone portion of TV-1106 sequence. D715 corresponds to D130 and N734 to N149 of the growth hormone sequence, respectively. These two sites in the growth hormone sequence had been reported previously in the art to be susceptible to the modifications observed in this study. In a later set of experiments, an additional deamidation site (N684) and an additional isomerization site (D692/697) were identified in more stressed samples.

### Example 4

### TV-1106 Formulation Candidate Selection

Long term stability was evaluated for 3 formulation candidates at 50 mg/mL and 100 mg/mL TV-1106 protein concentrations. The formulations in glass cartridge were compared to the same formulation in glass vial because the presence of silicone oil required for cartridge functionality in glass cartridge may potentially decrease TV-1106 stability. Table 18 describes the compositions of each candidate formulation.

TABLE 18
Compositions of TV-1106 formulation candidates

| Formulation | TV-1106, | Histidine, | Phosphate, | NaCl, | Mannitol, | Trehalose, | |
|---|---|---|---|---|---|---|---|
| code | mg/mL | mM | mM | mM | mM | mM | pH |
| 50-PMT-C | 50 | 0 | 20 | 0 | 180 | 60 | 6.0 |
| 100-PMT-C | 100 | 0 | 20 | 0 | 180 | 60 | 6.0 |
| 50-HN-C | 50 | 20 | 0 | 140 | 0 | 0 | 6.5 |
| 50-HNMT-C | 50 | 20 | 0 | 50 | 25 | 75 | 6.5 |
| 100-PMT-V | 100 | 0 | 20 | 0 | 180 | 60 | 6.0 |
| 50-HN-V | 50 | 20 | 0 | 140 | 0 | 0 | 6.5 |
| 50-HNMT-V | 50 | 20 | 0 | 50 | 25 | 75 | 6.5 |

C: formulation stored in glass cartridge;
V: formulation stored in glass vial

Deamidation and isomerization values at three months for the PMT formulation at two concentrations of TV-1106 and the HN formulation are shown in Table 19.

TABLE 19
Deamidation data for PMT and HN formulations

| | | | | Peptide mapping | | |
|---|---|---|---|---|---|---|
| | Conditions/treatment | | | with MS (%) | | |
| | | Storage | | D715 | N734 | ISOQUANT ® |
| Formulation | ° C. | time | pH | iso | d-N | % |
| PMT, 50 | 2-8 | 3 m | 6.0 | 2.9 | 2.2 | 2 |
| mg/mL | 25 | 3 m | 6.0 | 25.4 | 16 | 20 |
| protein | 32 | 3 m | 6.0 | 39.6 | 36.5 | 40 |
| PMT, 100 | 25 | 3 m | 6.0 | 26.7 | 18.6 | 21 |
| mg/mL | | | | | | |
| HN, 50 | 2-8 | 3 m | 6.5 | 0.8 | 4.8 | 4 |
| mg/mL | 25 | 3 m | 6.5 | 21.1 | 36.5 | 37 |
| protein | 32 | 3 m | 6.5 | 32.1 | 61.8 | 52 |

As demonstrated in Table 19, deamination of N734 and isomerization of D715 is temperature and pH dependent, but is not concentration dependent.

Performance of all formulation candidates were evaluated with SEC-HPLC, RP-HPLC, iCE280, ISOQUANT®, and relative potency after stored at 2-8° C. for up 12 months.

Isoaspartate levels of TV-1106 candidate formulations (PMT, HN, HNMT) at 2-8° C. determined by ISOQUANT® are illustrated in FIG. 2. Isoaspartate levels of formulations in PMT buffer, at both 50 and 100 mg/mL protein concentrations, was ˜60% less than those in HN or HNMT buffer at 12 months. Formulations stored in glass cartridges performed as well as formulations stored in vials.

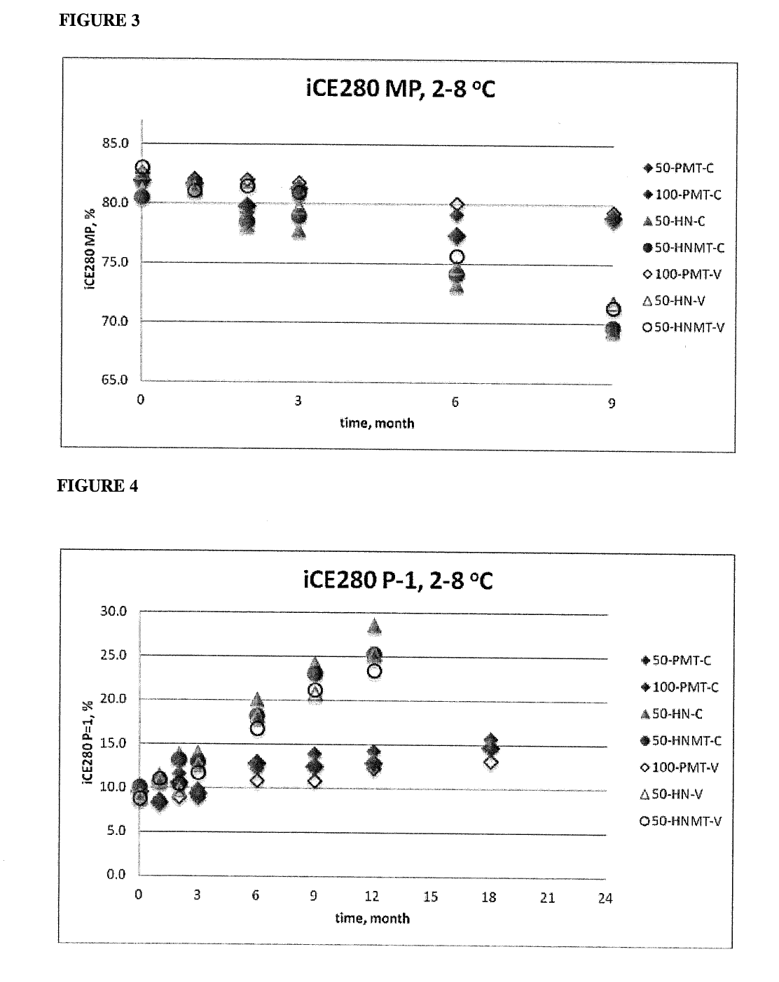

Charge heterogeneity of TV-1106 candidate formulations (PMT, HN, HNMT) at 2-8° C. is shown in FIG. 3. Charge heterogeneity is more stable in PMT buffer, at both 50 and 100 mg/mL protein concentrations, than in HN or HNMT buffer at 12 months. Formulations stored in glass cartridges performed as well as formulations stored in vials.

P-1 charge variant of TV-1106 candidate formulations (PMT, HN, HNMT) at 2-8° C. is shown in FIG. 4. Increase in P-1 charge variant in PMT buffer, at both 50 and 100 mg/mL protein concentrations, was at least about 50% lower than in HN or HNMT buffer at 12 months. Formulations stored in glass cartridges performed as well as formulations stored in vials.

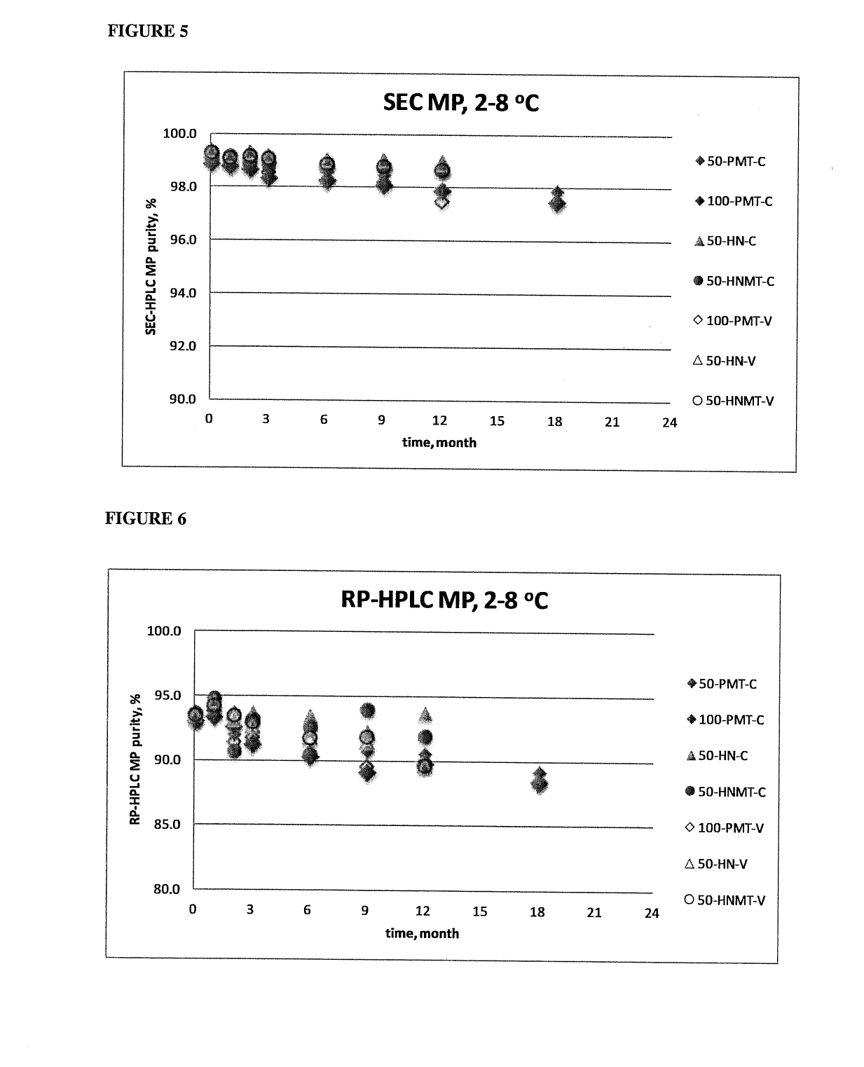

Purity measured by SEC-HPLC of TV-1106 candidate formulations (PMT, HN, HNMT) at 2-8° C. is shown in FIG. 5. Purity measure by SEC-HPLC was adequate in PMT buffer, at both 50 and 100 mg/mL protein concentrations, as in HN or HNMT buffer at 12 months. Formulations stored in glass cartridges performed as well as formulations stored in vials.

Purity measured by RP-HPLC of TV-1106 candidate formulations (PMT, HN, HNMT) at 2-8° C. is shown in FIG. 6. Purity measured by RP-HPLC was adequate in PMT buffer, at both 50 and 100 mg/mL protein concentrations, as in HN or HNMT buffer at 12 months. Formulations stored in glass cartridges performed as well as formulations stored in vials.

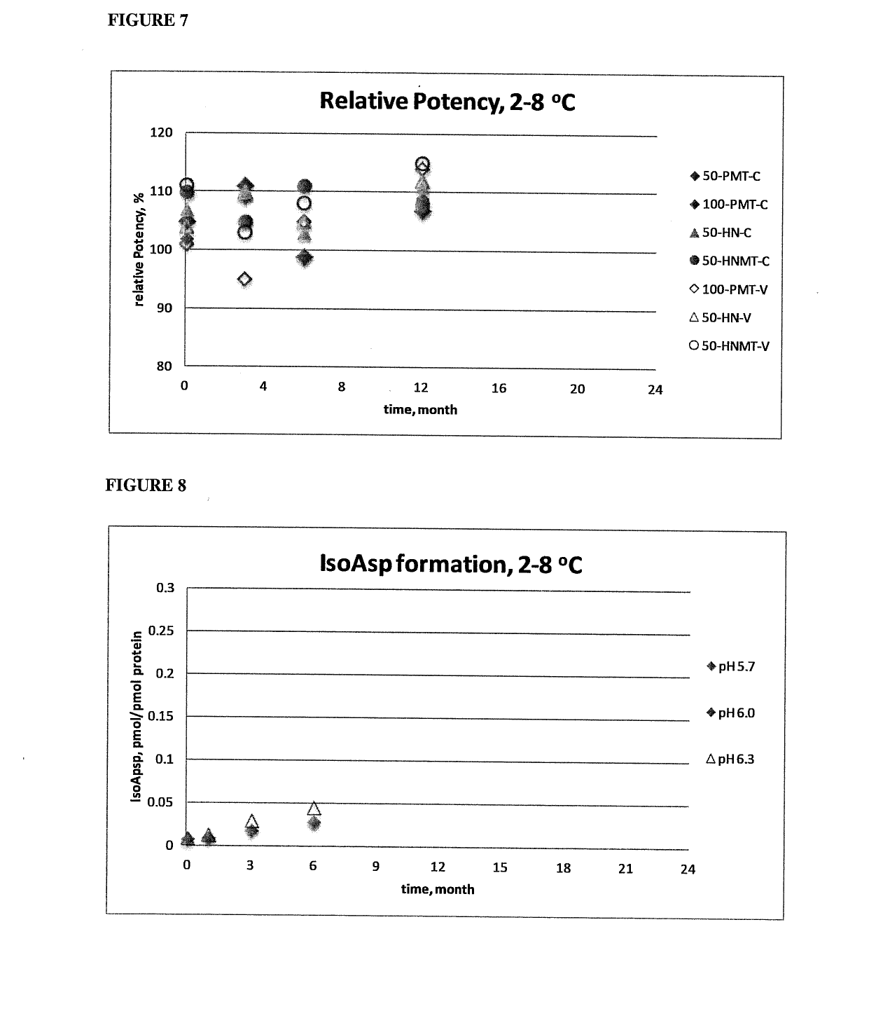

Relative potency of TV-1106 candidate formulations (PMT, HN, HNMT) at 2-8° C. are shown in FIG. 7. There was no potency loss in all formulation candidates. Formulations stored in glass cartridges performed as well as formulations stored in vials.

The results suggested that PMT formulation outperformed HN and HNMT formulation in both cartridge and vial at both 50 and 100 mg/mL TV-1106 concentrations. PMT formulation significantly reduced TV-1106 deamidation/isomerization while maintaining full potency and acceptable purities measured by SEC-HPLC and RP-HPLC. The PMT formulation showed only 2% deamidation/isomerization after 3 months of storage at 2-8° C. (Table 19), whereas 2.3% deamidation/isomerization was observed in a sample of TV-1106 formulated in 10 mM sodium phosphate, 200 mM mannitol, 60 mM trehalose, 0.01% polysorbate 80, pH 7.2 after only one month of storage at 2-8° C.

PMT formulation was selected and continued to be further evaluated beyond 12 months.

### Example 5

### pH Bracket and Control of Target TV-1106 Formulation

Long term stability was evaluated for target TV-1106 formulation (100 mg/mL TV-1106 in 20 phosphate, 180 mM mannitol, 60 mM trehalose, pH 5.7, 6.0 and 6.3). Table 20 describes the compositions of each formulation.

TABLE 20
Compositions of TV-1106 formulations in pH bracket study

| Formulation | TV-1106, | Phosphate, | Mannitol, | | |
|---|---|---|---|---|---|
| code | mg/mL | mM | mM | Trehalose, mM | pH |
| pH 5.7 | 100 | 20 | 180 | 60 | 5.7 |
| pH 6.0 | 100 | 20 | 180 | 60 | 6.0 |
| pH 6.3 | 100 | 20 | 180 | 60 | 6.3 |

Performance of each formulation was evaluated with SEC-HPLC, RP-HPLC, iCE280, ISOQUANT®, and relative potency was evaluated after being stored at 2-8° C. at the indicated time points.

Isoaspartate levels of the TV-1106 target formulations at pH 5.7, 6.0, and 6.3 at 2-8° C. is shown in FIG. 8. Isoaspartate levels of formulations at pH 5.7 and 6.0 was similar while slightly increased at pH 6.3.

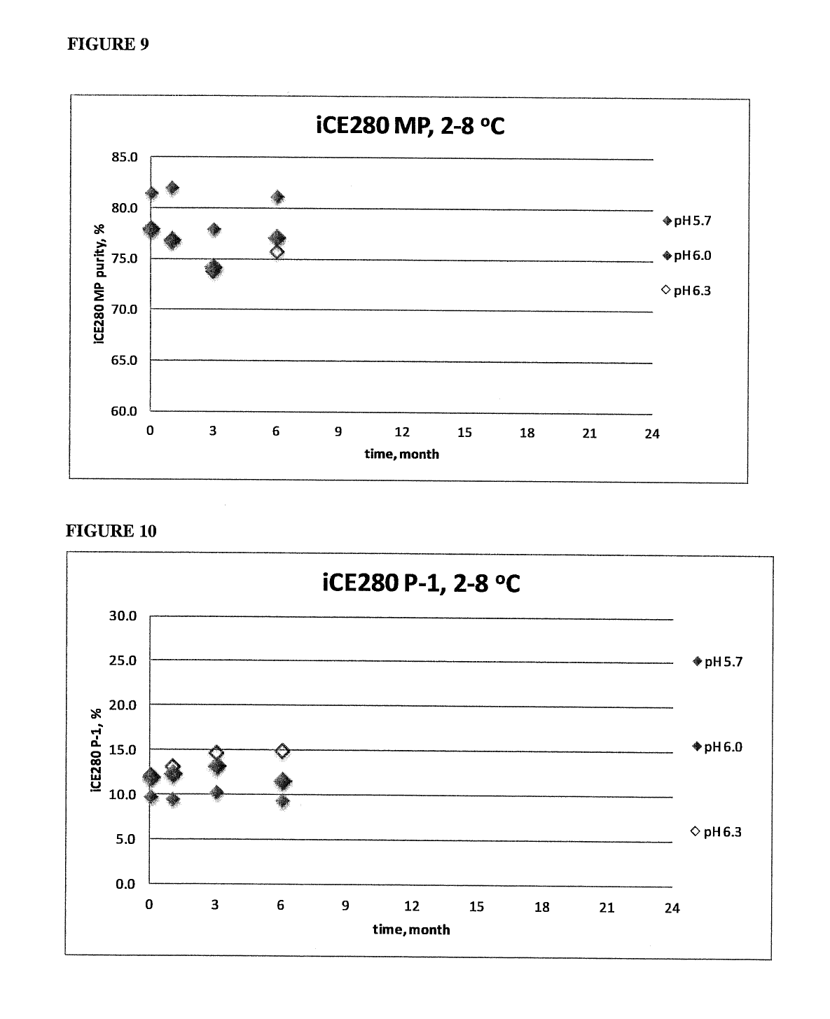

Charge heterogeneity of TV-1106 target formulations at pH 5.7, 6.0, and 6.3 at 2-8 CC is shown in FIG. 9. Loss of Charge heterogeneity appeared comparable among all three pHs.

P-1 charge variant of TV-1106 target formulations at pH 5.7, 6.0, and 6.3 at 2-8° C. is shown in FIG. 10. Increase of P-1 charge variant at pH 5.7 and 6.0 was similar, while slightly increased at pH 6.3.

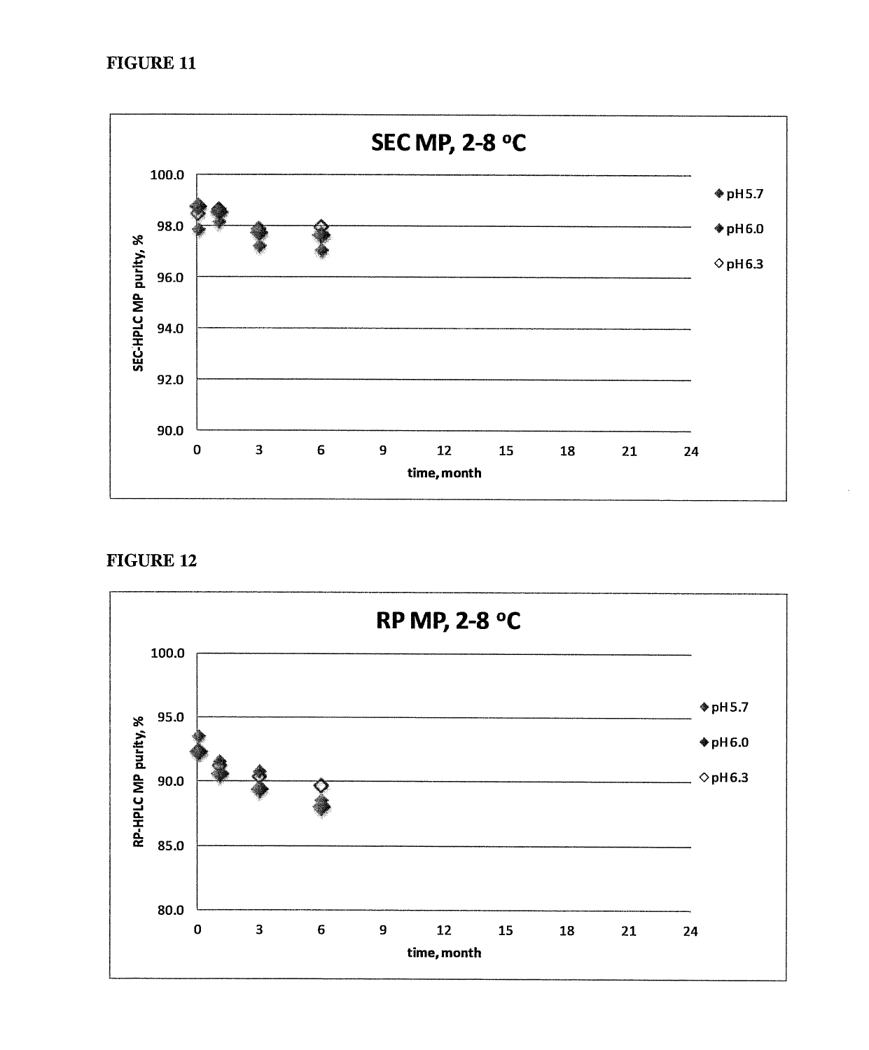

Purity measured by SEC-HPLC of TV-1106 target formulations at pH 5.7, 6.0, and 6.3 at 2-8° C. is shown in FIG. 11. Loss of SEC-HPLC main peak appeared similar at pH 5.7 and 6.0, while slightly less at pH 6.3.

Purity measured by RP-HPLC of TV-1106 target formulations at pH 5.7, 6.0, and 6.3 at 2-8° C. is shown in FIG. 12. Loss of RP-HPLC main peak appeared similar at pH 5.7 and 6.0, while slightly less at pH 6.3.

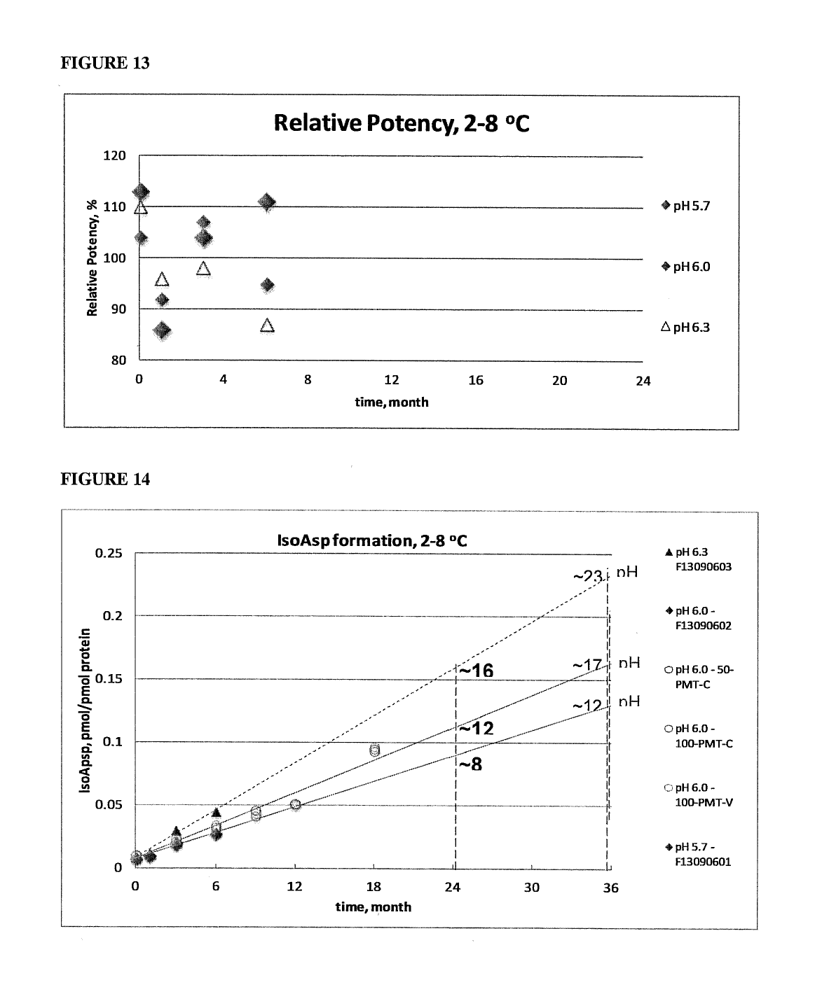

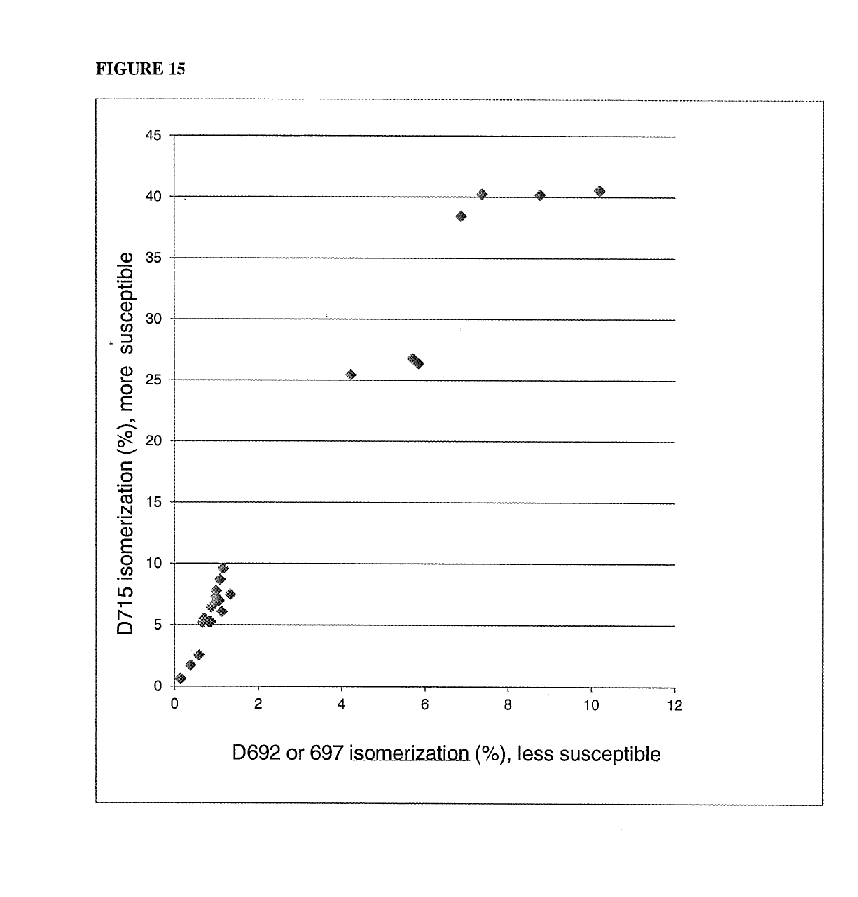

Relative potency of TV-1106 target formulations at pH 5.7, 6.0, and 6.3 at 2-8° C. is shown in FIG. 13. There was no potency loss in formulations at all three pHs.

A projection of isoaspartate levels in TV-1106 in the PMT, 100 mg/mL formulation at the end of 24 and 36 months of storage at 2-8° C. was created using 18 month real-time data and is shown in FIG. 14.

The results suggested that target TV-1106 formulation at pH 6.0 demonstrated a balanced stabilization against deamidation/isomerization and against the loss of SEC-HPLC, RP-HPLC purities and main charge variant. The slightly different yet close stability trending between pH 5.7 pH 6.3 showed that pH target 6.0 provided an acceptable stability range and manufacturing control strategy.

### Example 6

### Deamidation of Asparagine Residues and Isomerization of Aspartate Residues in TV-1106 PMT Formulation Compared to Commercial Formulations of rhGH

The storage stability of a 100 mg/mL formulation of TV-1106 comprising 20 mM sodium phosphate, 180 mM mannitol, 60 mM trehalsoe, and residual amounts of polysorbate 80 (1.2 mg of TV-1106) at a pH of 6.0 (PMT) was compared to the storage stability of marketed rhGH formulations.

TABLE 21
TV1106 Deamidation Profile Comparison With Approved rhGH Products
of Most Susceptible Deamidation/Isomerization Sites

| | | | D130/D715 isomerization | | | N149/N734 deamidation | | |
|---|---|---|---|---|---|---|---|---|
| | | | | T + 3 | T + 6 | | T + 3 | T + 6 |
| Product | pH | Temp. | T + 0 | month | month | T + 0 | month | month |
| Nutropin ® | 6.0 | 2-8° C. | 7.78 | 8.71 | 9.59 | 6.42 | 7.02 | 7.53 |
| AQ (liquid) | | 25° C. | | na | 40.19 | | na | 47.06 |
| Saizen ® | 5.6-6.6 | 2-8° C. | 5.29 | 6.46 | 7.28 | 6.31 | 6.74 | 8.42 |
| (reconstituted | | 25° C. | 5.52 | 25.45 | 38.45 | 6.39 | 38.14 | 62.36 |
| from lyophilized | | | | | | | | |
| powder) | | | | | | | | |
| Norditropin ® | No data | 2-8° C. | 5.25 | 6.10 | 7.49 | 6.36 | 7.19 | 9.12 |
| (liquid) | | 25° C. | | 26.39 | 40.53 | | 24.99 | 47.49 |
| TV-1106 | 6.0 | 2-8° C. | 0.63 | 1.73 | 2.55 | 1.63 | 2.12 | 3.32 |
| | | 25° C. | | 26.79 | 40.24 | | 19.59 | 40.39 |

TABLE 22
TV1106 Deamidation Profile Comparison With Approved
rhGH Products of Less Susceptible Deamidation Sites
The average deamidation/isomerization rate for the
tested formulations is shown in Table 23.

| | | | D107/692 or 112/697 | | | N99/N684 | | |
|---|---|---|---|---|---|---|---|---|
| | | | isomerization | | | deamidation | | |
| | | | | T + 3 | T + 6 | | T + 3 | T + 6 |
| Product | pH | Temp. | T + 0 | month | month | T + 0 | month | month |
| Nutropin ® | 6.0 | 2-8° C. | 1.00 | 1.09 | 1.16 | 0.36 | 0.34 | 0.18 |
| AQ | | 25° C. | | na | 8.75 | | na | 2.20 |
| Saizen ® | 5.6-6.6 | 2-8° C. | 0.80 | 0.88 | 0.99 | 0.30 | 0.36 | 0.33 |
| | | 25° C. | 0.71 | 4.22 | 6.86 | 0.27 | 1.14 | 2.00 |
| Norditropin ® | No data | 2-8° C. | 0.86 | 1.13 | 1.33 | 0.31 | 0.23 | 0.46 |
| | | 25° C. | | 5.83 | 10.18 | | 1.01 | 2.14 |
| TV-1106 | 6.0 | 2-8° C. | 0.14 | 0.38 | 0.58 | 0.23 | 0.18 | 0.22 |
| | | 25° C. | | 5.70 | 7.37 | | 0.86 | 1.40 |

TABLE 23
Average deamidation/isomerization rates

| | Average | |
|---|---|---|
| | deamidation/ | |
| | isomerization rate | |
| | at 2-8° C., | |
| | %/Month by | |
| Product | ISOQUANT ® | SD |
| Nutropin ® AQ | 0.356 | 0.051 |
| Saizen ® | 0.283 | 0.024 |
| Norditropin ® | 0.250 | 0.024 |
| TV-1106 (100 mg/mL | 0.267 | 0.067 |
| PMT) | | |

### Conclusions

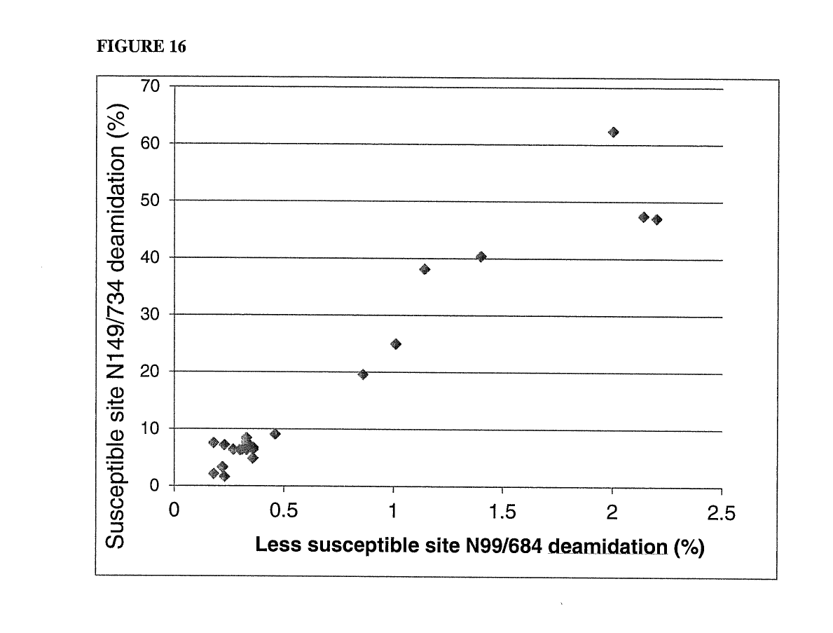

D715 and N734 are the two most susceptible sites for deamidation/isomerization, consistent with literature documentation of rhGH deamidation profile. The two additional deamidation/isomerization sites, N684 and D692 or 697, identified in TV-1106 were identified in approved rhGH products as well (corresponding to N99 and D107 or 112 of hGH). These two sites are less susceptible to deamidation/isomerization as the deamidation/isomerization levels are low and detectable under stressed storage condition or physiological condition (FIGS. 16-17.)

Lower levels of deamidation/isomerization were generally observed in the TV-1106 formulation compared to the commercial formulations of rhGH.

### Example 7

### Process for Producing TV-1106 with Increased Yield

Osborn 2002 describes producing TV-1106 by fermentation using the yeast strain BXP10, which is a genetically modified form of the yeast S. cerevisiae laboratory strain AH22 that had been optimized for the production of recombinant human serum albumin with minimal post-transcriptional modification.

Upon scale up from a 10 L culture volume to a culture volume of greater than 200 L, yield of TV-1106 is decreased. Addition of polysorbate 80 to the culture medium increases the yield of TV-1106 recovered from the culture medium.

### Discussion

Current hGH treatment is limited by problems with compliance (Haverkamp et al. 2008; Cutfield et al. 2011). Specific rates of noncompliance, while difficult to determine with certainty, have been reported in the art to range from 34 to 85 percent, with most studies reporting 75 percent or less compliance (Haverkamp et al. 2008). Noncompliance reduces the efficacy of hGH treatment in promoting linear growth, and results in significant waste of funding for hGH treatments (Cutfield et al. 2011). Noncompliance is especially high in chronic diseases which do not present discomfort, such as GHD (Haverkamp et al. 2008). It has been found that complex treatment regimes are one of the causes of noncompliance and treatment simplification will increase patient compliance (Haverkamp et al. 2008).

U.S. patent application publication No. US 2014-0162954 A1 discloses a lyophilized formulation of TV-1106 that requires reconstitution with sterile water for injection (WFI) prior to use. The present invention provides a liquid composition of TV-1106 that does not require reconstitution and may be stored in liquid form via cartridges, vials, injection pens, syringes, etc. and can be directly administered to the patient without reconstitution.

The storage stable liquid formulations of TV-1106 described herein have significant advantages over the previously described lyophilized composition, including increased convenience (since patients will not have to reconstitute the formulation before administration), increased dosage accuracy, and ability to package the formulation in ready to use cartridges or injection pen devices for patient self-administration. The storage stable liquid formulations of TV-1106 herein can be used, for example, to treat any disease or disorder for which rhGH therapy is currently administered, e.g., growth failure due to growth hormone deficiency in children, or adult growth hormone deficiency. The storage stable liquid formulations of TV-1106 be used to treat both naïve and experienced users. For pediatric patients, the dosing of the formulation may be weight based in conjunction with a biomarker, e.g., IGF, check. In some embodiments, up to 100 mg of TV-1106 may be administered per administration to pediatric patients. In some embodiments, up to 50 mg of TV-1106 may be administered per administration to adult patients. The storage stable liquid formation may be provided to the patient in an injection pen/device, syringe, or vial.

It was discovered that select formulations of TV-1106 described herein, e.g. the PMT formulation at pH 6.0, decrease the rate of deamidation at asparagine residues and isomerization at aspartate residues in TV-1106 without significantly decreasing protein stability. pH selection was determined to be critical to the storage stability of the fusion protein. In particular, it was discovered that lowering the pH of the formulation from pH 7.2 of the formulation of US 2014-0162954 A1 significantly reduced deamidation and isomerization rates without significant loss of native TV-1106 structure. Moreover, the formulations described herein provide significantly higher protein concentrations (compared to marketed rhGH formulations and the TV-1106 described in US 2014-0162954 A1) without significant increases in protein aggregation. High concentration formulations of TV-1106 provide greater packaging, shipping, and storage efficiency, and may help reduce injection site discomfort by allowing for smaller administration volumes, resulting in greater patient compliance. For example, high concentration formulations, e.g., 100 mg/mL of TV-1106, allow for administration volumes of 50 μl to 1 mL in some embodiments of the invention.

Compared to currently marketed liquid formulations of rhGH, such as Nutropin® AQ and Norditropin®, the PMT formulation showed lower levels of isomerization and deamidation at the most susceptible deamidation and isomerization sites following storage, even at the same pH. Thus, the formulations described herein represent an advance over known liquid formulations of rhGH in terms of protein purity and stability while satisfying the clinical need for a storage stable liquid formulation of TV-1106.

### REFERENCES

Ablinger, E. et al., “Effect of protamine on the solubility and deamidation of human growth hormone.” International Journal of Pharmaceutics, Vol. 427, Issue 2, 10 May 2012, pp. 209-216.
Ablinger, E. et al., “Stabilization of Human Growth Hormone Against Deamidation by Interaction with Strongly Basic Protamines from Salmon.” Conference abstract PDD04, Presented at the 8th Central European Symposium on Pharmaceutical Technology, Sep. 16-18, 2010, Graz, Austria, published in Scientia Pharmaceutics 2010, 78:593.
Bossart J., Benchmarking Drug Delivery—Defining Product Benefit and Value. Drug Dev. & Delivery, 2005 February; 5(2): 22.
Brown, Larry R. “Commercial challenges of protein drug delivery.” Expert opinion on drug delivery 2.1 (2005): 29-42.
Carroll P V, Christ E R, Bengtsson B A, Carlsson L, Christiansen J S, Clemmons D, Hintz R, Ho K, Laron Z, Sizonenko P, Sonksen P H, Tanaka T and Thorne M. Growth hormone deficiency in adulthood and the effects of growth hormone replacement: a review. Growth Hormone Research Society Scientific Committee. J Clin Endocrinol Metab. 1998; 83:382-95.
Cazares-Delgadillo J, Ganem-Rondero A, Kalia Y N. “Human growth hormone: New delivery systems, alternative routes of administration, and their pharmacological relevance.” Eur J Pharm Biopharm 2011; 78: 278-288.
Chou, Danny K., et al, “Effects of TWEEN® 20® and TWEEN® 80® on the stability of Alubtropin During Agitation.” Journal of Pharmaceutical Sciences, Vol. 94, No. 6, June 2005, pp. 1368-1381.
Cutfield, Wayne S., et al. “Non-compliance with growth hormone treatment in children is common and impairs linear growth.” PloS one 6.1 (2011): e16223. Emedicine [homepage on the Internet]. Overview on Growth Hormone Deficiency. Available from emedicine.medscape.com/article/120767-overview#a0104 (accessed Jul. 10, 2014).
Genentech Press Release, Jun. 1, 2004, Genentech and Alkermes Announce Decision to Discontinue Commercialization of NUTROPIN DEPOT™, available at www.gene.com/gene/news/press-releases/display.do?method=detail&id=7447 (accessed Oct. 17, 2012).
Genentech Press Release, Dec. 23, 1999, FDA Approves NUTROPIN DEPOT™, First Long-Acting Dosage Form of Recombinant Growth Hormone for Growth Hormone Deficiency in Children, available at www.gene.com/gene/news/press-releases/display.do?method=print&id=4879 (accessed Oct. 17, 2012).
Haverkamp, Fritz, et al. “Observations of nonadherence to recombinant human growth hormone therapy in clinical practice.” Clinical Therapeutics 30.2 (2008): 307-316.
Johnson, Brett A., “Formation of Isoaspartate at Two Distinct Sites during in Vitro Aging of Human Growth Hormone*.” The Journal of Biological Chemistry, Vol. 264, Issue of August 25, pp. 14262-14271 (1989).
Klibanski A, et al. ALBUTROPIN™ in Adults with Growth Hormone Deficiency. 1st Joint Meeting of the Growth Hormone Research Society and the International Society for Insulin-like Growth Factor Research. Oct. 8, 2002. Poster 18.
Kober M M. Letter to Robert L. Garnick, Ph.D., Jul. 28, 2000, available at www.fda.gov/downloads/Drugs/GuidanceComplianceRegulatorylnformation/EnforcementActivitiesbyFDA/WarningLettersandNoticeofViolationLetterstoPharmaceuticalCompanies/UCM166042.pdf (accessed Oct. 17, 2012).
NUTROPIN DEPOT™ Label, 2004, available at www.accessdata.fda.gov/drugsatfda_docs/label/2004/21075s0081b1.pdf (accessed Oct. 17, 2012).
Osborn, Blaire L., et al. “Albutropin: a growth hormone-albumin fusion with improved pharmacokinetics and pharmacodynamics in rats and monkeys.” European journal of pharmacology 456.1 (2002): 149-158.
Osterberg L and Blaschke T. “Adherence to medication.” N Engl J Med 2005; 353: 487-497.
Reiter, Edward O., et al., “A multicenter study of the efficacy and safety of sustained release GH in the treatment of naïve pediatric patients with GH deficiency.” Journal of Clinical Endocrinology & Metabolism 86.10 (2001): 4700-06.
Robinson, N. E. and Robinson, A. B., “Deamidation of human proteins.” Proc. Natl. Acad. Sci. USA 98, pp. 12409-12413 (Oct. 23, 2001).
Rosenfeld R., Bakker B. Compliance and persistence in pediatric and adult patients receiving growth hormone therapy; Endocr Pract 2008; 14:143-154.
Saenger P., “Current status of biosimilar growth hormone.” Int J Pediatr Endocrinol 2009; 2009:370329 (1-6).
Somatropin Biopartners EPAR summary for the public, European Medicines Agency 2013.
Teshima, Glen, et al., “Isolation and Characterization of a Succinimide Variant of Methionyl Human Growth Hormone.” The Journal of Biological Chemistry, Vol. 266, Issue of July 25, pp. 13544-13547 (1991).

Therapeutic Proteins: Methods and Protocols, C M. Smales, D. C. James, ed., Humana Press, 2005
Yang, Seung Yun, et al. “Single-file diffusion of protein drugs through cylindrical nanochannels.” ACS nano 4.7 (2010): 3817-22.

1. A stable liquid pharmaceutical composition comprising an albumin-human growth hormone (hGH) fusion protein whose amino acid sequence is set forth as SEQ ID NO: 1 and a buffer, wherein the stable liquid pharmaceutical composition has a pH range of 5.5-6.5.

2. The stable liquid pharmaceutical composition of claim 1, characterized in that
i) 0.01-0.15 moles of isoaspartate residues are present per mole of fusion protein upon storage of the stable liquid pharmaceutical composition for 12 months at 2-8° C., and/or
ii) less than 0.25, less than 0.20, loss than 0.15, or less than 0.10 moles of isoasrpartate residues are present per mole of fusion protein upon storage of the stable liquid pharmaceutical composition for 24 months at 2-8° C., and/or
iii) less than 0.25, less than 0.20, or less than 0.15 moles of isoaspartate residues are present per mole of fusion protein upon storage of the stable liquid pharmaceutical composition for 36 months at 2-8° C.

3-5. (canceled)

6. The stable liquid pharmaceutical composition of claim 1, characterized in that
i) 1-5% of the fusion protein has an isoaspartate residue at amino acid position D715 upon storage of the stable liquid pharmaceutical composition for 6 months at 2-8° C., and/or
ii) less than 45% of the fusion protein has an isoaspartate residue at amino acid position D715 upon storage of the stable liquid pharmaceutical composition for 6 months at 20° C.

7-8. (canceled)

9. The stable liquid pharmaceutical composition of claim 1, characterized in that
i) 1-5% of the fusion protein has an isoaspartate residue at amino acid position N734 upon storage of the stable liquid pharmaceutical composition for 6 months at 2-8° C., and/or
ii) less than 45% of the fusion protein has an isoaspartate residue at amino acid position N734 upon storage of the stable liquid pharmaceutical composition for 6 months at 25° C.

10-11. (canceled)

12. The stable liquid pharmaceutical composition of claim 1, characterized in that i) 0.1-1% of the fusion protein has an isoaspartate residue at amino acid position D692, amino acid position D697, or amino acid positions D692 and D697 upon storage of the stable liquid pharmaceutical composition for 6 months at 2-8° C., and/or
ii) less than 10%, less than 9%, or less than 8% of the fusion protein has an isoaspartate residue at amino acid position D692, amino acid position D697, or amino acid positions D692 and D697 upon storage of the stable liquid pharmaceutical composition for 6 months at 25° C.

13-14. (canceled)

15. The stable liquid pharmaceutical composition of claim 1, characterized in that
i) 0.1-0.4% of the fusion protein has an isoaspartate residue at amino acid position N684 upon storage of the stable liquid pharmaceutical composition for 6 months at 2-8° C., and/or
ii) less than 2.0, loss than 1.7%, or less than 1.5% of the fusion protein has an isoaspartate residue at amino acid position N684 upon storage of the stable liquid pharmaceutical composition for months at 25° C.

16-18. (canceled)

19. A stable liquid pharmaceutical composition comprising (i) an albumin-human growth hormone (hGH) fusion protein whose amino acid sequence is set forth as SEQ ID NO: 1, and (ii) a pharmaceutically acceptable excipient comprising a buffer, in which 0.01-0.15 moles of isoaspartate residues are present per mole of fusion protein after storage for 12 months at 2-8° C.

20. The stable liquid pharmaceutical composition of claim 19, in which
i) less than 0.10, less than 0.09, less than 0.08, less than 0.07, less than 0.06, or less than 0.05 moles of isoaspartate residues are present per mole of fusion protein after storage for 12 months at 2-8° C., and/or
ii) less than 0.25, less than 0.20, less than 0.15, or less than 0.10 moles of isoaspartate residues are present per mole of fusion protein after storage for 24 months at 2-8° C., and/or
iii) less than 0.25, less than 0.20, or less than 0.15 moles of isoaspartate residues are present per mole of fusion protein after storage for 36 at 2-8° C.

21-22. (canceled)

23. A stable liquid pharmaceutical composition of claim 1, in which 1-5% of the fusion protein has an isoaspartate residue at amino acid position D715 after storage for 6 months at 2-8° C.

24. (canceled)

25. The stable liquid pharmaceutical composition of claim 23, in which
i) 1-5% of the fusion protein has an isoaspartate residue at amino acid position N734 after storage for 6 months at 2-8° C., and/or
ii) 0.1-1% of the fusion protein has an isoaspartate residue at amino acid position D692, amino acid position D697, or amino acid positions D692 and D697 after storage for 6 months at 2-8° C., and/or
iii) 0.1-0.4% of the fusion protein has an isoaspartate residue at amino acid position N684 upon storage of the stable liquid pharmaceutical composition for 6 months at 2-8° C.

26-30. (canceled)

31. The stable liquid pharmaceutical composition of claim 23, in which 1-5% of the fusion protein has an isoaspartate residue at amino acid position D715 after storage for 6 months at 2-8° C., 1-5% of the fusion protein has an isoaspartate residue at amino acid position N734 after storage for 6 months at 2-8° C., 0.1-1% of the fusion protein has an isoaspartate residue at amino acid positions D692 and D697 after storage for 6 months at 2-8° C., and 0.1-0.4% of the fusion protein has an isoaspartate residue at amino acid position N684 upon storage of the stable liquid pharmaceutical composition for 6 months at 2-8° C.

32. (canceled)

33. The stable liquid pharmaceutical composition of claim 1, wherein the buffer comprises
i) phosphate, and/or
ii) histidine, and/or
iii) mannitol, and/or
iv) trehalose.

34-45. (canceled)

46. The stable liquid pharmaceutical composition of claim 1, wherein
i) the stable liquid pharmaceutical composition has an osmolality from 250 to 350 mOsm/kg, and/or
ii) the pH is 5.7-6.3, and/or
iii) the concentration of the fusion protein is 2-200 mg/mL.

47-48. (canceled)

49. The stable liquid pharmaceutical composition of claim 1, further comprising
i) sodium chloride, and/or
ii) polysorbate 80.

50-54. (canceled)

55. The stable liquid pharmaceutical composition of claim 1, wherein the purity of the fusion protein decreases by 2.5% or less after incubation at 25° C. for 72 hours.

56. The stable liquid pharmaceutical composition of claim 2, wherein the presence of isoaspartate residues is determined by (i) an assay which detects isoaspartate residues via protein isoaspartyl methyltransferase (PIMT)-catalyzed generation of S-adenosyl homocysteine (SAH), (ii) isoelectric focusing, (iii) ion-exchange chromatography, or (iv) protein mapping and mass spectrometry.

57. A package comprising the stable liquid pharmaceutical composition of claim 1 and a container, wherein the container is a cartridge, a vial, a pre-filled syringe, and infusion pump, or an injection pen.

58-63. (canceled)

64. A process for preparing a stable liquid pharmaceutical composition comprising an albumin-human growth hormone (hGH) fusion protein whose amino acid sequence is set forth as SEQ ID NO: 1, the process comprising:
a. determining the number of moles of isoaspartate residues present per mole of fusion protein in a batch of the fusion protein; and
b. preparing the stable liquid pharmaceutical composition from the batch only if less than 0.05, less than 0.04, less than 0.03, or less than 0.02 moles of isoaspartate residues are present per mole of fusion protein in the batch.

65-66. (canceled)

67. A process for validating a batch of a stable liquid pharmaceutical composition comprising an albumin-human growth hormone (hGH) fusion protein of claim 1 whose amino acid sequence is set forth as SEQ ID NO: 1 for distribution, the process comprising:
a. determining the number of moles of isoaspartate residues present per mole of fusion protein in a sample of the batch; and
b. validating the batch for distribution only if the number of moles of isoaspartate residues per mole of fusion protein is below a predetermined threshold number.

68-76. (canceled)

77. A method of treating a human patient in need of growth hormone therapy by periodically administering to the human patient for more than two weeks an effective amount of the stable liquid pharmaceutical composition of claim 1.

78-90. (canceled)

91. A process for producing an albumin-human growth hormone (hGH) fusion protein of claim 1 whose amino acid sequence is set forth as SEQ ID NO: 1 comprising culturing a recombinant cell capable of expressing the fusion protein in a culture medium comprising a polysorbate, and isolating the fusion protein from the culture medium.

92-105. (canceled)


SEQUENCE LISTING (1 sequence)

SEQ ID NO: 1 - protein, 776 aa, Artificial Sequence
  Amino acid sequence of the albumin-hGH fusion protein TV-1106
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF